(19) United States
(12) Patent Application Publication
(10) Pub. No.: US 2025/0285287 A1
(43) Pub. Date: Sep. 11, 2025

(54) ANCHOR POINTS-BASED IMAGE SEGMENTATION FOR MEDICAL IMAGING

(71) Applicant: Genentech, Inc., South San Francisco, CA (US)

(72) Inventors: Seyed Mohammadmohsen HEJRATI, Stanford, CA (US); Miao ZHANG, Foster City, CA (US)

(21) Appl. No.: 19/215,102

(22) Filed: May 21, 2025

Related U.S. Application Data
(63) Continuation of application No. PCT/US23/81042, filed on Nov. 24, 2023.
(60) Provisional application No. 63/427,639, filed on Nov. 23, 2022.

(51) Int. Cl. G06T 7/12; A61B 5/00; G06T 7/136; G16H 50/20

(52) U.S. Cl. CPC G06T 7/12; A61B 5/742; A61B 5/748; G06T 7/136; G16H 50/20; G06T 2200/24; G06T 2207/10081; G06T 2207/10088; G06T 2207/10101; G06T 2207/10104; G06T 2207/10108; G06T 2207/10116; G06T 2207/10132; G06T 2207/20081; G06T 2207/20092; G06T 2207/30004

(57) ABSTRACT

A method that is implemented by one or more computer devices and that includes receiving an image associated with a portion of anatomy of a subject. Boundary points are extracted for an initial boundary associated with a corresponding region of pixels in the image. The boundary points are evaluated according to a sequential order to select anchor points. The evaluating includes determining that a current boundary point being evaluated is a next anchor point when at least one perpendicular distance computed for a portion of the initial boundary located between a previous anchor point and the current boundary point with respect to a line extending between the previous anchor point and the current boundary point is greater than a selected threshold. An anchor point image is generated for display in a graphical user interface on a display device. The anchor point image includes anchor indicators that represent the anchor points.

### CROSS-REFERENCE TO RELATED APPLICATIONS

This application is a continuation of International Application No. PCT/US2023/081042 filed on Nov. 24, 2023, which is related to and claims the benefit of the priority date of U.S. Provisional Application No. 63/427,639, filed Nov. 23, 2022, entitled “Anchor Points-Based Image Segmentation for Medical Imaging,” each of which is incorporated herein by reference in its entirety.

### TECHNICAL FIELD

This application relates generally to analyzing images, and, more particularly, to methods and systems that include image analysis to generate anchor indicators on a graphical user interface that enable efficient automatic and/or semi-automatic adjustments to the boundaries of anatomical regions of interest identified using image segmentation.

### BACKGROUND

Medical imaging typically involves visualizing and analyzing digitized images to ascertain whether variations occurring in tissues are due to disease, toxicity, and/or natural processes. Specifically, medical imaging may rely upon one or more image analysis tasks to convert raw image data into a qualitative understanding of the tissues. For example, the one or more image analysis tasks generally include image enhancement, image segmentation, image feature extraction, and, finally, image classification. Some instances of image analysis may include identifying certain regions of tissues that appear normal, diseased, or correspond to one or more other similar regions of interest. For example, by identifying the regions of the tissues that appear normal or diseased and quantifying the area, shape, or texture of these regions of the tissues, computational-based methods can perform in minutes what would otherwise require hours of laborious tasks performed by a pathologist, a number of graders, or other scientific or medical experts.

Image segmentation generally includes partitioning a digitized image into different regions of pixels. In some instances, class labels may be assigned to each of the different regions of pixels (e.g., normal region, diseased region, a region of concern, or a region of interest). In some cases, the class labels may be predicted utilizing a machine-learning model trained to recognize characteristics of regions of pixels (e.g., features) in the digitized image. For instance, in one example, as part of the drug discovery and development process, the machine-learning model-based image segmentation may be relied upon to accurately classify regions of pixels of a scan of a tissue corresponding, for example, to tumor bed, stroma, or healthy tissue for purposes of suitable treatment identification. In another instance, the machine-learning model-based image segmentation may be relied upon to accurately classify regions of pixels of a scan of a patient's retina corresponding, for example, to the individual retinal layers of the patient's retina and/or one or more fluid pockets. After identifying a suitable treatment, such as through discovery of new pharmaceuticals, these new pharmaceuticals may then be subject to regulatory approval requirements by governmental or regulatory institutions. For example, the approval procedure may include a pathologist, a human grader, a central reading center (CRC), or other medical or scientific professionals reviewing not only the efficacy of the new pharmaceuticals themselves, but also the efficacy and accuracy of the means and methods by which efficacy was ascertained and/or the new pharmaceuticals were developed. However, because the image segmentation task may not alone render sharp, distinct boundaries between the different regions of pixels of a medical scan, for example, the class labels corresponding to the different regions of pixels may be inaccurate or imprecise.

### SUMMARY

In one or more embodiments, a method includes receiving an image associated with a portion of anatomy of a subject. The method includes extracting a plurality of boundary points for an initial boundary associated with a corresponding region of pixels in the image, wherein the plurality of boundary points is associated with a sequential order for a selected two-dimensional plane corresponding to the image. The method includes evaluating the plurality of boundary points according to the sequential order to select a plurality of anchor points from the plurality of boundary points. The evaluating comprises determining that a first boundary point in the plurality of boundary points is a first anchor point. The evaluating comprises determining that a current boundary point of the plurality of boundary points being evaluated is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold. The method includes generating an anchor point image for display in a graphical user interface on a display device, wherein the anchor point image includes a plurality of anchor indicators that represent the plurality of anchor points.

In one or more embodiments, a method includes receiving a plurality of images associated with a portion of anatomy of a subject. The method includes performing segmentation using the plurality of images and a segmentation model to generate a plurality of segmented images. The method includes extracting, for each segmented image of the plurality of segmented images, a plurality of boundary points for each initial boundary of a set of initial boundary associated with each segmented image. The plurality of boundary points is associated with a sequential order for a selected two-dimensional plane corresponding to the plurality of images. The method includes evaluating, for each segmented image of the plurality of segmented images, the plurality of boundary points for each initial boundary of the set of initial boundary associated with each segmented image according to the sequential order to select a plurality of anchor points from the plurality of boundary points for each initial boundary of the set of initial boundary identified in each image. The evaluating comprises: determining that a first boundary point in the plurality of boundary points is a first anchor point; determining that a last boundary point in the plurality of boundary points is a last anchor point; and determining that a current boundary point of the plurality of boundary points being evaluated is a next anchor point in the plurality of anchor points when at least one perpendicular distance of a set of perpendicular distances computed for a corresponding set of middle boundary points with respect to a line extending between a previous anchor point and the current boundary point is greater than a selected threshold. The method includes generating an anchor point image for display in a graphical user interface on a display device, wherein the anchor point image includes a plurality of anchor indicators that represent the plurality of anchor points and a plurality of line segments connecting the plurality of anchor indicators.

In one or more embodiments, a system includes one or more computing devices that include one or more non-transitory computer-readable storage media including instructions; and one or more processors coupled to the one or more storage media. The one or more processors are configured to execute the instructions to receive an image associated with a portion of anatomy of a subject. The one or more processors are configured to execute the instructions to extract a plurality of boundary points for an initial boundary associated with a corresponding region of pixels in the image, wherein the plurality of boundary points is associated with a sequential order for a selected two-dimensional plane corresponding to the image. The one or more processors are configured to execute the instructions to evaluate the plurality of boundary points according to the sequential order to select a plurality of anchor points from the plurality of boundary points. The evaluation includes determining that a first boundary point in the plurality of boundary points is a first anchor point. The evaluation includes determining that a current boundary point of the plurality of boundary points being evaluated is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold. The one or more processors are configured to execute the instructions to generate an anchor point image for display in a graphical user interface on a display device, wherein the anchor point image includes a plurality of anchor indicators that represent the plurality of anchor points.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations.

FIG. 1A is a block diagram of an image analysis system in accordance with one or more example embodiments.

FIG. 1B is a block diagrams of the image processor from FIG. 1A described in further detail in accordance with one or more example embodiments.

FIG. 2A-2B together are a flowchart of a process for generating adjustment data in accordance with one or more example embodiments.

FIGS. 3A-3B together are flowchart of a process for evaluating boundary points in accordance with one or more example embodiments.

FIG. 4 is a workflow diagram of a process for evaluating a plurality of boundary points for possible inclusion in a plurality of anchor points in accordance with one or more embodiments.

FIG. 5 is a diagram illustrating how a perpendicular distance is computed in accordance with one or more example embodiments.

FIG. 6 is an illustration of an image displayed in a graphical user interface in accordance with one or more example embodiments.

FIG. 7 is an illustration of a segmented image displayed in a graphical user interface in accordance with one or more example embodiments.

FIG. 8 is an illustration of an image with identified boundaries displayed in a graphical user interface in accordance with one or more example embodiments.

FIG. 9 is an illustration of an image displayed with boundary indicators in a graphical user interface in accordance with one or more example embodiments.

FIG. 10 is an illustration of an image with anchor indicators displayed in a graphical user interface in accordance with one or more example embodiments.

FIG. 11 is an illustration of an image displayed with boundary indicators in a graphical user interface in accordance with one or more example embodiments.

FIG. 12 is an illustration of an image with anchor indicators displayed in a graphical user interface in accordance with one or more example embodiments.

FIG. 13 is an illustration of an image displayed with boundary indicators in a graphical user interface in accordance with one or more example embodiments.

FIG. 14 is an illustration of an image with anchor points displayed in a graphical user interface in accordance with one or more example embodiments.

FIG. 15 is an illustration of an image displayed with boundary indicators in a graphical user interface in accordance with one or more example embodiments.

FIG. 16 is an illustration of an image with anchor points displayed in a graphical user interface in accordance with one or more example embodiments.

FIG. 17 is an illustration of a graphical user interface transforming the display of a boundary to anchor indicators in accordance with one or more example embodiments.

FIG. 18 is an illustration of a graphical user interface transforming the display of a boundary to anchor indicators in accordance with one or more example embodiments.

FIG. 19 is an illustration of a graphical user interface transforming the display of a boundary to anchor indicators in accordance with one or more example embodiments.

FIG. 20 is an illustration of the graphical user interface from FIG. 19 showing a change in anchor point density in accordance with one or more example embodiments.

FIG. 21 is a block diagram illustrating an example of a computing system, in accordance with one or more example embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The embodiments described herein take into account that in certain cases, currently available image analysis techniques may not provide a desired level of accuracy for use in the screening, diagnosing, and/or managing the treatment of diseases. Further, embodiments described herein take into account that certain rules, regulations, and/or requirements associated with clinical trials, governmental institutions, and/or regulatory institutions may mandate a level of human intervention in order to correct for imprecise data generated using automated techniques. For example, relying entirely on machine learning models for diagnosing or analyzing the treatment of diseases based on medical images may not be fully embraced by certain members or groups of the medical community or the regulatory community and in some cases, may not be allowed by the regulatory community. Thus, it is desirable to improve the accuracy of information generated using machine learning models.

For example, machine learning models may be used to segment various images (e.g., medical images including, for example, OCT images of the retina or MRI images of the brain). For example, AI-based semantic segmentation may be used to classify each pixel as belonging to one of various selected categories. A selected category may be, for example, a retinal fluid layer, brain tissue, lung tissue, or some other anatomical structure or feature. This process can be used to generate segmented images that identify the regions of pixels that belong to the different categories. Boundaries of these regions may also be identified.

However, in some cases, the identification of these regions and their boundaries may not have the desired level of accuracy. In some cases, it may be desirable for human users to review the segmented regions and/or their boundaries to adjust the boundaries so as to more accuracy reflect the actual anatomical regions. Accordingly, it may be desirable to have methods and systems that enable fast, efficient, and accurate adjustment of these boundaries based on user input. New images generated with the newly adjusted boundaries may be used as training data to retrain the machine learning models to more accurately segment the regions and boundaries.

Thus, the embodiments described herein provide automatic and semi-automatic methods, and computer-readable media for improving the accuracy of the data using machine learning models. For example, the embodiments described herein provide an image analysis system that may be used to generate adjustment data that can be used to adjust images or that includes adjusted images in which the boundaries of certain anatomical regions of interest identified on these images more accurately reflect the actual anatomical region of interest.

In one or more embodiments, a segmented image may identify multiple regions of pixels, each region of pixels being classified as belonging to a selected category. The segmented image may be associated with a portion of anatomy of a subject (e.g., an eye, retina, brain, lung, chest, leg, etc.). A region of pixels may correspond to a fluid layer, a pathological element, a type of tissue, a lobe, or some other type of anatomical structure or feature.

A set of initial boundaries associated with the regions of pixels may be identified using the segmented image. In some cases, these boundaries are identified in the segmented image. In other cases, these boundaries may be computed from the segmented image. An initial boundary that is associated with a region of pixels may be, for example, but is not limited to, an outer border, an inner border, a lower boundary, an upper boundary, a side boundary, a combination thereof, or some other type of boundary. A plurality of boundary points may be extracted for an initial boundary. The plurality of boundary points is associated with a sequential order for a reference two-dimensional (2D) plane corresponding to the image. The plurality of boundary points may be evaluated according to the sequential order to select a plurality of anchor points from the plurality of boundary points. This evaluation may include, for example, automatically determining that a first boundary point in the plurality of boundary points is a first anchor point. This evaluation may include, for example, automatically determining that a last boundary point in the plurality of boundary points is a last anchor point. This evaluation may include, for example, automatically determining that a current boundary point of the plurality of boundary points being evaluated is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold.

The selected threshold may be selected to provide a desired density of anchor points with a desired level of complexity of curvature, grooves, texture, and/or contour in the boundary that is represented by the anchor points. A higher threshold may be selected to reduce the density (number of) anchor points and reduce the complexity of curvature, grooves, texture, and/or contour in the boundary that is captured. In contrast, a lower threshold may be selected to increase the density (number of) anchor points and increase the complexity of curvature, grooves, texture, and/or contour in the boundary that is captured.

A graphical user interface is generated in which an anchor point image is displayed. The anchor point image may include, for example, the original image on which segmentation was performed and a plurality of anchor indicators that represent the plurality of anchor points. The plurality of anchor indicators may be graphical indicators that are controllable. For example, a human user (e.g., a medical professional or expert, a human pathologist, a human grader, a reading center, or other type of human user) may move the anchor indicator to change the position of the corresponding anchor point and thereby, the corresponding boundary.

By viewing the anchor indicators on the original image, the human user can make a determination about where the boundary generated via machine learning techniques can be adjusted to more accurately reflect the actual corresponding boundary associated with the subject's anatomy. In this manner, a direction and/or curvature of a boundary may be controlled based on the user input.

The anchor points are selected in order to reduce overall computing resources that would otherwise be spent by providing the user with an option to adjust every pixel that forms the boundary of a region of interest. By using the anchor indicators that represent the anchor points, the user can focus on those critical points along the boundary that affect curvature, contour, texture, etc. and the system can more easily and efficiently update the boundary based on fewer inputs without sacrificing accuracy. In some embodiments, the segmented image may be adjusted to reclassify (or reassign) various pixels based on the new/revised boundary.

The image analysis system may process the user input and automatically adjust the position of the corresponding anchor points to thereby automatically adjust the previously identified boundaries. The image analysis system may generate, for example, adjusted images with new boundaries that can be used as new labeled data for supervised and/or semi-supervised training and/or retraining of machine learning models. In this manner, the machine learning models used to segment images may be better trained to more accurately perform segmentation.

Using anchor points and anchor indicators that represent those anchor points in the manner described herein may enable adjustment/correction of segmented images in a manner that reduces the overall time and resources needed to make these adjustments/corrections, without sacrificing accuracy. Because the anchor indicators represent only a subset of the boundary points extracted for a given boundary, adjustments to the overall boundary may be more efficiently made without the user needing to adjust each boundary point (pixel) of that boundary. Further, with fewer anchor indicators, there are fewer overall adjustment computations that need to be made without sacrificing overall accuracy. Thus, generating anchor points and a graphical user interface that displays controllable/movable anchor indicators that represent the anchor points may improve the overall functioning of the image analysis system described herein and may reduce overall consumption of computing resources.

### II. Example Architecture for an Image Analysis System

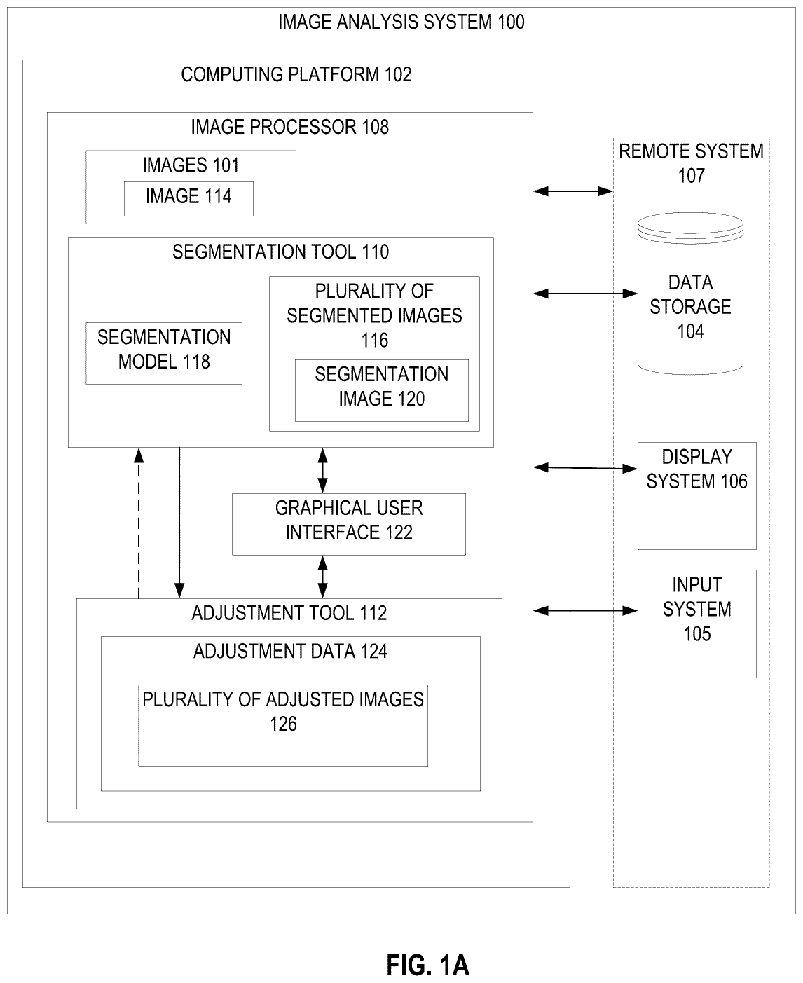

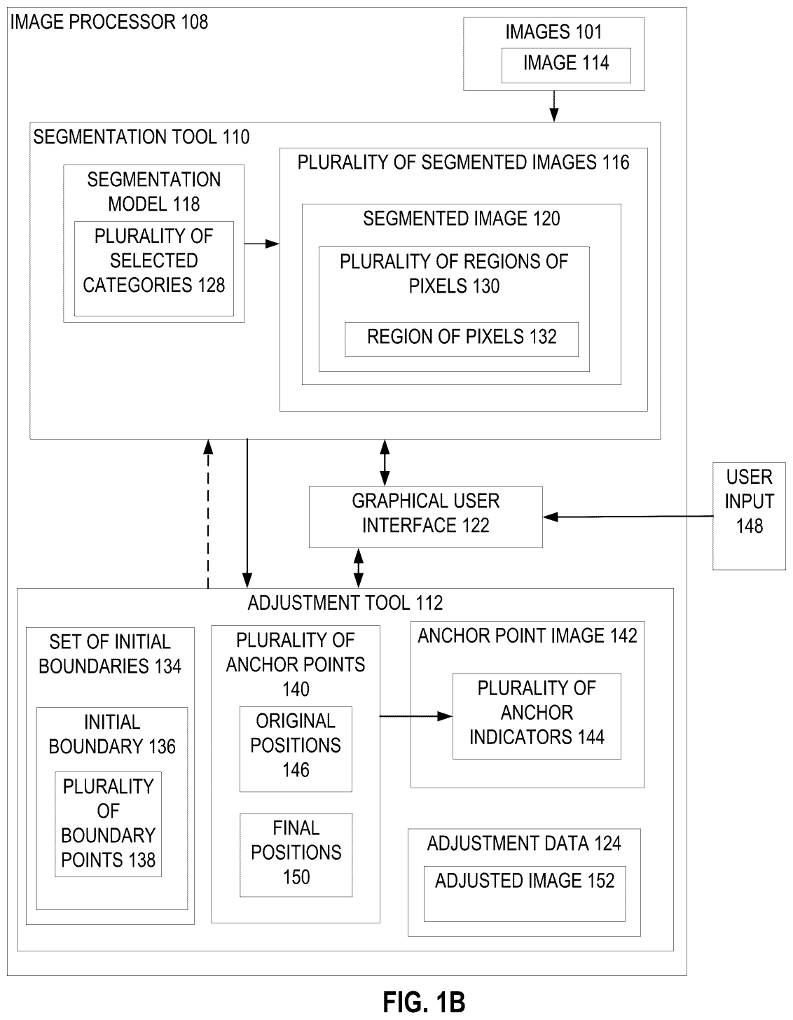

FIG. 1A is a block diagram of an image analysis system 100 in accordance with one or more example embodiments. The image analysis system 100 may be used to analyze a plurality of images such as, for example, without limitation, images 101. Each image of the plurality of images 101 may be an image of a portion of anatomy of a subject. For example, the images 101 may be medical images. Accordingly, the image analysis system may also be referred to as a medical imaging analysis system. Further, image analysis system 100 may be used to analyze images using image segmentation and may therefore also be referred to as an image segmentation and analysis system or a medical image segmentation and analysis system.

The image analysis system 100 may be used for training and using machine learning models to perform segmentation of images (e.g., OCT images) more accurately and efficiently, for providing evaluation, detection, diagnosis, and/or treatment of patients with disease, or a combination thereof. Image analysis system 100 can include a computing platform 102, a data storage 104, an input system 105, and a display system 106. In one or more embodiments, data storage 104, input system 105, display system 106, or any combination thereof may be part of remote system 107 that is located remotely with respect to computing platform 102.

Computing platform 102 may take various forms. In one or more embodiments, computing platform 102 includes a single computer (e.g., server or computer system), multiple computers (e.g., servers) in communication with each other (e.g., via one or more wired, one or more wireless, and/or one or more optical communication links), a smart television, and/or any combination thereof. In other examples, computing platform 102 takes the form of a cloud computing platform, a mobile computing platform (e.g., a smartphone, a tablet, a laptop), or a combination thereof.

In some embodiments, the computing platform 102 may include, but is not limited to, a cloud-based computing architecture suitable for hosting, servicing, and/or interfacing with one or more hardware, software, and/or firmware modules or tools executing on remote system 107. In one or more embodiments, computing platform 102 may include a Platform as a Service (PaaS) architecture, a Software as a Service (SaaS) architecture, an Infrastructure as a Service (IaaS) architecture, a Compute as a Service (CaaS) architecture, a Data as a Service (DaaS) architecture, a Database as a Service (DBaaS) architecture, or other similar cloud-based computing architecture (e.g., “X” as a Service (XaaS)).

Hardware in computing platform 102 may include, for example, but is not limited to, a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various medical profile data and making one or more decisions based thereon. Computing platform 102 may include software (e.g., instructions running/executing on one or more processors), firmware (e.g., microcode), or some combination thereof.

Data storage 104, input system 105, and/or display system 106, may each be in communication with computing platform 102 (e.g., using one or more wired, wireless, optical, and/or other type of communications links). In some examples, data storage 104, input system 105 display system 106, or any combination thereof may be considered part of or otherwise integrated with computing platform 102. Thus, in some examples, computing platform 102, data storage 104, and display system 106 may be separate components in communication with each other, but in other examples, some combination of these components may be integrated together. Data storage 104 may include, for example, one or more data stores such as, one or more databases.

Input system 105 may include one or more input devices for receiving user input. Input system 105 may include, for example, but is not limited to, a mouse, a touchpad, a keyboard, a touchscreen, a keypad, a joystick, a virtual reality input device, a plurality of arrow keys, or a combination thereof that allows a user to enter user input. Display system 106 may include one or more display devices such as, but not limited to, a monitor, a television, a screen, or some other type of display device. A device such as a touchscreen may function as both an input device for input system 105 and a display device for display system 106.

The image analysis system 100 includes image processor 108, which may be implemented using hardware, software, firmware, or a combination thereof. In one or more embodiments, image processor 108 is implemented in computing platform 102. In some embodiments, a first portion (e.g., hardware, firmware, and/or software) of image processor 108 is implemented in computing platform 102 and a second portion (e.g., hardware, firmware, and/or software) of image processor 108 is implemented in remote system 107.

Image processor 108 may include, for example, segmentation tool 110 and adjustment tool 112, each of which may be implemented using hardware, software, firmware, or a combination thereof. In one or more embodiments, image processor 108 receives the images 101 for processing. In some embodiments, the images 101 may be received from data storage 104 and/or some other type of data store (e.g., cloud storage) in communication with computing platform 102. The images 101 may capture a portion of the anatomy of a subject (e.g., a patient). For example, the image 101 may capture a subject's retina, one or more bones, brain, one or more lungs, one or more kidneys, liver, bladder, heart, other tissue, or a combination thereof.

Further, the images 101 may be medical images (e.g., medical scans, medical diagnostics images) that include optical coherence tomography (OCT) images (e.g., OCT volumes, OCT B-scans), magnetic resonance imaging (MRI) images, computed tomography (CT) images, X-ray images, ultrasound images, positron-emission tomography (PET) images, single-photon emission computerized tomography (SPECT) images, or some other type of 2D/3D image that captures the portion of anatomy. Image 114 may be one example of the images 101. Image 114 may take the form of an OCT image such as, for example, an OCT B-scan, which can be used to capture and render retinal layer depth. An OCT B-scan refers to the cross-sectional image where the amplitudes of reflections are represented in a gray scale or a false-color scale. In some cases, OCT B-scans may be referred to as brightness scans. In other embodiments, image 114 is an MRI image, a CT image, or some other type of medical imaging scan.

In one or more embodiments, images 101 are generated after processing of the initial images generated by a medical imaging system (e.g., CT device, MRI device, OCT scanner, etc.). For example, the initial images generated by the medical imaging system may be filtered, scaled, resized, cropped, and/or preprocessed in some other manner to generate the images 101.

After image processor 108 receives the images 108, segmentation tool 110 may process the received images 101 to generate a plurality of segmented images 116, which may be 2D or 3D images. In one or more embodiments, segmentation tool 110 may include segmentation model 118 that can receive each of the images 101 as an input and generate a corresponding segmented image to form the segmented images 116. Segmentation model 118 includes a machine learning model such as, for example, without limitation, a deep learning model, which may itself include any number of or combination of models (e.g., one or more neural networks). For example, the deep learning model may include any number of neural networks including for example, one or more convolutional neural networks (CNNs). In one or more embodiments, segmentation model 118 includes a deep learning model that performs artificial intelligence (AI)-based semantic segmentation. Accordingly, segmentation model 118 may, in some cases, be referred to as a semantic segmentation model, an AI segmentation model, or a deep learning (DL) segmentation model. Semantic segmentation is a pixel by pixel segmentation in which each pixel is classified (or labeled) as belonging to a category.

To perform AI-based semantic segmentation of an image such as image 114 to generate a corresponding segmented image 120, segmentation model 118 groups the pixels of the image 114 into a plurality of categories. For example, segmentation model 118 may label or otherwise classify each pixel in image 114 as belonging to one of these categories (e.g., two, three, four, five, size, seven, eight, nine, ten, or some other number of categories).

In one or more embodiments, segmentation tool 110 or another portion of image processor 108 may display segmented image 120 in graphical user interface (GUI) 122 on display system 106. Graphical user interface 122 may be generated by image processor 108 for display on display system 106 and may allow a user to interact with graphical user interface 122. Segmented image 120 may be displayed in graphical user interface 122 such that each group of pixels that belongs to a different category may be represented with a different graphical indicator or feature to distinguish each group of pixels. For example, different colors, shading, highlighting, patterns, labels, text, and/or other types of graphical indicators or graphical features may be used to distinguish the different groups of pixels for the different categories. As one specific example, each different category may be represented by the corresponding pixels for each different category having a different color in graphical user interface 122.

In one or more embodiments, each group of pixels belonging to a different category may be referred to as a “segment” in the segmented image 120. A segment may be a continuous or discontinuous grouping of pixels. For example, a segment may be formed by one or more separate regions of pixels, each of which is labeled as belonging to a same category. Alternatively, a segment may be an individual region of pixels such that a particular grouping of pixels may be represented in the segmented image 120 by one or more segments. In this manner, segmentation model 118 may perform one or more image segmentation processes (e.g., semantic image segmentation) to segment regions of pixels included in the image 114.

Adjustment tool 112 may receive the segmented images 116 from segmentation tool 110 for processing. Adjustment tool 112 may process segmented images 116 to generate adjustment data 124. Adjustment data 124 may be data providing adjusted or corrected data that can be used to adjust (or correct) segmented images 116 so that the adjusted segmented images 116 can be used as an input for a model. For example, adjustment data 124 may include a plurality of adjusted images 126 that can be used to retrain segmentation model 118. The adjusted images 126, which may be 2D or 3D images, may be used to train/retrain a different machine learning model. The adjusted images 126 may be used as input for another model or algorithm to generate additional information about images 101, generate outputs based on images 101 (e.g., screening, diagnosis or treatment management outputs). The adjustments may be to the definition of or position/shape/size of a region of interest. The types of adjustments that may be made are described further with respect to FIG. 1B.

FIG. 1B is a block diagrams of image processor 108 from FIG. 1A described in further detail in accordance with one or more example embodiments. In FIG. 1B, segmentation tool 110 and adjustment tool 112 of image processor 108 are described in further detail.

As previously described, segmentation model 118 groups the pixels of each image of the images 101 into a plurality of selected categories 128. Selected categories 128 may include 1, 2, 3, 4, 5, 6, 7, 8, 9, 10, 11, 12, 13, 14, 15, 16, 17, 18, 19, 20, or some other number of categories. Selected categories 128 may be selected based on one or more regions of interest. Segmentation model 118 can be used to identify a single anatomical structure or feature of interest, segmentation model 118 may classify each pixel in an image as representing that anatomical structure/feature of interest or not (e.g., background). Segmentation model 118 may also be used to identify multiple features of interest such as fluid layers. Segmentation model 118 may also be used to identify multiple features of interest such as fluid layers. Selected categories 128 may include, for example, background, a normal region, a diseased region, a region of concern, a fluid layer, a pathological element, a specific type of tissue (e.g., brain tissue, lung tissue, bone tissue, etc.), a specific type of organ, some other type of anatomical structure or feature, or a combination thereof.

For example, segmentation model 118 may label or otherwise classify each pixel in image 114 as belonging to one of selected categories 128. In one or more embodiments, each group of pixels belonging to a different category may be referred to as a “segment” in the segmented image 120. A segment may be a continuous or discontinuous grouping of pixels. For example, a segment may be formed by one or more separate regions of pixels, each of which is labeled as belonging to a same category. Alternatively, a segment may be an individual region of pixels such that a particular grouping of pixels (e.g., all pixels for a selected category) may be represented in the segmented image 120 by one or more segments. In this manner, segmentation model 118 may perform one or more image segmentation processes (e.g., semantic image segmentation) to segment regions of pixels included in the image 114.

For example, segmentation tool 110 may process image 114 to generate segmented image 120, which includes a plurality of regions of pixels 130 in which all pixels in a selected region (e.g., region of pixels 132) of the plurality of regions of pixels 130 belong to a same selected category of selected categories 128. One region of pixels may form the entirety of a segment in segmented image 120 or multiple regions of pixels may form a segment in segmented image 120.

Adjustment tool 112 may receive segmented images 116 (including segmented image 120) from segmentation tool 110 for processing. For each of segmented images 116, adjustment tool 112 identifies one or boundaries for one or more regions of interest. A region of interest may be, for example, one region of the plurality of regions of pixels 130. In one or more embodiments, segmented image 120 received at adjustment tool 112 includes an identification or indication of set of initial boundaries 134. For example, segmented image 120 may be annotated to identify set of initial boundaries 134. In other embodiments, adjustment tool 112 determines set of initial boundaries 134 based on the segments in segmented image 120.

For example, for segmented image 120, adjustment tool 112 processes segmented image 120 to identify a set of initial boundaries 134. Set of initial boundaries 134 includes one or more initial boundaries such as, for example, initial boundary 136. Initial boundary 136 is associated with a corresponding region of pixels, which may be region of pixels 132. For example, initial boundary 124 may include the outer border of region of pixels 132, an inner border of region of pixels 132, an upper boundary of region of pixels 132, a lower boundary of region of pixels 132, a selected boundary between region of pixels 132 and another adjacent region of pixels, or some other type of boundary that is related to or provides information about the anatomical structure(s) or feature(s) represented by region of pixels 132.

For each boundary of set of initial boundaries 134, adjustment tool 112 extracts a plurality of boundary points. For example, adjustment tool 112 extracts a plurality of boundary points 138 for initial boundary 136. In one or more embodiments, boundary points 138 may be the pixels that form initial boundary 136. For example, boundary points 138 may be X,Y positions or X,Y,Z positions (e.g., in pixel units or in some other units used to identify pixel locations) for each pixel that forms initial boundary 136. In other embodiments, boundary points 138 include every n^{th} pixel that forms initial boundary 136, where n may be (e.g., 1, 2, 3, 4, 5, etc.).

Boundary points 138 may be associated with a sequential order. In one or more embodiments, the sequential order is associated with a selected two-dimensional (2D) plane or a selected direction in the X,Y,Z coordinate system. For example, when image 114 and/or segmented image 120 are 2D images, the 2D plane may be the 2D plane corresponding to either or both of these images. In this example, the sequential order is left to right across the 2D plane such that the sequential order begins with the boundary point (pixel) from the leftmost column of pixels to the rightmost column.

When image 114 and/or segmented image 120 is a 3D image, the sequential order may be as described above but for each 2D plane that is parallel to a selected direction or reference 2D plane in the X,Y,Z coordinate system. Thus, for a 3D image, boundary points 138 may include a different group of boundary points for each selected plane/slice that is parallel to the reference 2D plane. The sequential order may be left to right across each of these individual selected planes/slices.

Adjustment tool 112 then evaluates boundary points 138 according to the sequential order to select a plurality of anchor points 140 from the plurality of boundary points. In other words, anchor points 140 includes a subset of boundary points 138. Anchor points 140 is selected such that the line segments that connect the anchor points approximate a curvature of initial boundary 136 within selected tolerances. The selected tolerances are selected such that the line segment connecting any pair of anchor points does not over-linearize the curvature of the corresponding portion of initial boundary 136. For example, the linearization of a curved portion of initial boundary 136 by the line extending between a pair of anchor points may be measured by the greatest perpendicular distance between a line segment extending between the pair of anchor points and initial boundary 136. A description of how boundary points 138 may be evaluated to select the subset that form anchor points 140 is described further in Section III (Example Methodologies of Image Analysis) below.

Once anchor points 140 has been determined, adjustment tool 112 may generate an anchor point image 142 for display on graphical user interface 122. Anchor point image 142 may include, for example, original image 114 from which segmented image 120 was generated, with a plurality of anchor indicators 144 overlaid on original image 114 at positions corresponding to the original positions 146 of anchor points 140.

In one or more embodiments, each anchor indicator in anchor indicators 144 may be a graphical indicator that is movable or controllable via user input 148. In other words, anchor indicators 144 may be manipulable by a user. For example, adjustment tool 112 may receive user input 148 via graphical user interface 122 (e.g., entered by a user via input system 105 described in FIG. 1A) that causes the position of one or more of anchor indicators 144 to change on anchor point image 142. User input 148 may include a user moving an anchor indicator (e.g., dragging an anchor indicator from its current position to a new position). User input 148 may include a user selecting (e.g., clicking, etc.) a new position for a particular anchor indicator. The user entering user input 148 may be, for example, a medical professional or medical expert such as, for example, but not limited to, a human pathologist, a human grader, a reading center, or other appropriate user.

In one or more embodiments, when anchor point image 142 is a 3D image displayed in 3D form, graphical user interface 122 may display anchor point image 142 in a manner that allows the user to control which cross-sectional view or slice of the 3D image is being displayed at a given point in time. These cross-sectional views or slices may be parallel to the reference 2D plane described above as being associated with boundary points 138 and determining the sequential order associated with boundary points 138.

Adjustment tool 112 processes user input 148 to update the positions of one or more of anchor points 140 such that the anchor points 140 have final positions 150. Final positions 150 include at least one position that is different from (e.g., has been adjusted) the respective original position of the original positions 146 of anchor points 140.

Adjustment tool 112 may use final positions 150 of anchor points 140 to generate adjustment data 124. Adjustment data 124 may include, for example, final positions 150 for anchor points, new positions for other ones of boundary points 138 computed based on final positions 150, adjusted image 152, or a combination thereof. Adjusted image 152 may be one example of an adjusted image of plurality of adjusted images 126. Adjusted image 152 may be, for example, image 114 in which initial boundary 136 has been adjusted to form a new boundary that is represented on adjusted image (e.g., via a line, colored line, a patterned line, text, highlighting, etc.).

In one or more embodiments, adjusted image 152 may be an adjusted version of segmented image 120 in which the pixels have been reclassified based on the final positions 150 of anchor points 140. For example, final positions 150 of anchor points 140 may be used to identify a new set of boundaries. The new set of boundaries may be used to reclassify (or reassign) one or more pixels of segmented image 120. For example, a new boundary may be used to reclassify (or reassign) one or more pixels that currently belong to a first category as belonging to second category of the selected categories 128. The adjusted image 152 may be referred to as an adjusted segmented image and may be used to form training input for segmentation model 118 to train/retrain segmentation model 118 (or another type of segmentation model).

While the above-described process has been described with respect to adjustment tool 112 generating anchor points 140 and adjustment data 124 for initial boundary 136, the same process may be used to adjust any other initial boundaries present in set of initial boundaries 134 such that adjustment data 124 accounts for these one or more other initial boundaries. For example, adjustment image 152 may identify the new boundary for each one of set of initial boundaries 134.

Further, while the above-described process has been described with respect to adjustment tool 112 processing segmented image 120 to generate adjusted image 152, the same process may be used to process each of segmented images 116 to generate additional adjustment data 124 (e.g., adjustment images 126). For example, the embodiments described herein may be used to generate adjusted images 126 in the form of adjusted segmented images.

In one or more embodiments, adjustment data 124 may be used to create newly labeled input (e.g., labeled image inputs) for the supervised or semi-supervised retraining of segmentation model 118. In other embodiments, adjustment data 124 may be used to create labeled image input for the training/retraining of some other type of machine learning algorithm. In still other embodiments, adjustment data 124 may be used as the input for or to create the input for another algorithm or model. This other algorithm or model may be used to, for example, without limitation, screen, diagnose, or manage treatment (e.g., predict treatment response, select treatment with highest predicted efficacy, etc.) for a selected disease, disorder, or group of diseases/disorders.

Using anchor points and anchor indicators that represent those anchor points in the manner described above to enable a user to correct the boundaries identified in segmented images 116 reduces the overall time and resources needed to adjust/correct segmented images 116 to form adjusted images 126. Because the anchor indicators represent only a subset of the boundary points extracted for a given boundary, adjustments to the overall boundary may be more efficiently made without the user needing to adjust each boundary point (pixel) of that boundary. Further, with fewer anchor indicators, there are fewer overall adjustment computations that need to be made without sacrificing overall accuracy. Thus, generating anchor points and graphical user interface 122 that displays controllable/movable anchor indicators that represent the anchor points may improve the overall functioning of image analysis system 100 and reduce overall consumption of computing resources.

With reference still to FIGS. 1A and 1B, the various data and images generated by segmentation tool 110 and adjustment tool 112 may be stored in data storage 104 for future use. For example, image processor 108 may store the final positions of anchor points determined for segmented image 120 (e.g., the final positions for anchor points for each initial boundary of initial boundaries 134, including the final positions 150 of anchor points 140 for initial boundary 136) in data storage 104. In some embodiments, image processor 108 may store anchor point image 142 with anchor indicators 144 at the positions corresponding to final positions 150 of anchor points 140 in data storage 104. In some embodiments, image processor 108 may store adjustment data 124 in data storage 104. In some embodiments, image processor 108 may store segmented images 116 in data storage 104. In some embodiments, image processor 108 may store segmented images 116 with the final positions of anchor points identified on each of the segmented images 116 (e.g., final positions 150 of anchor points 140 identified on segmented image 120), images 101 with the final positions of anchor points identified on each of the images 101 (e.g., final positions 150 of anchor points 140 identified on image 114), or both. In a similar manner, in some embodiments, image processor 108 may store the set of initial boundaries and/or their corresponding boundary points in association with each of the segmented images 116, images 101, or both.

With reference still to FIGS. 1A and 1B, in one or more embodiments, images 101 that are processed by image processor 108 may take the form of OCT images that the retina of subjects. In these examples, segmentation tool 110 may be used to perform retinal segmentation on these OCT images. Retinal segmentation includes the detection and identification of one or more retinal (e.g., retina-associated) elements in a retinal image. A retinal element may be comprised of at least one of a retinal layer element or a retinal pathological element. Detection and identification of one or more retinal layer elements may be referred to as layer element (or retinal layer element) segmentation. Detection and identification of one or more retinal pathological elements may be referred to as pathological element (or retinal pathological element) segmentation.

A retinal layer element may be, for example, a retinal layer or a boundary associated with a retinal layer. Examples of retinal layers include, but are not limited to, an internal limiting membrane (ILM) layer, a retinal nerve fiber layer, a ganglion cell layer, an inner plexiform layer, an inner nuclear layer, an outer plexiform layer, an outer nuclear layer, an external limiting membrane (ELM) layer, a photoreceptor layer(s), a retinal pigment epithelial (RPE) layer, a layer of RPE detachment, a Bruch's membrane (BM) layer, a choriocapillaris layer, a choroidal stroma layer, an ellipsoid zone (EZ), and other types of retinal layer. In some cases, a retinal layer may be comprised of one or more layers. As one example, a retinal layer may be an outer plexiform layer-Henle fiber layer (OPL-HFL). A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of an RPE (IB-RPE) detachment layer, an outer boundary of the RPE (OB-RPE) detachment layer, or another type of boundary.

A retinal pathological element may include, for example, fluid (e.g., a fluid pocket), cells, solid material, or a combination thereof that evidences a retinal pathology (e.g., disease or condition such as AMD or DME). For example, the presence of certain retinal fluids may be a sign of nAMD or DME. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, drusen, a development of fibrosis, and a disruption. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption.

Additionally, a retinal pathological element may include a characteristic or subtype of one of the fluids (e.g., IRF, SRF, fluid associated with PED), materials (e.g., HRM, SHRM, IHRM), lesions (e.g., HRF, SHRM lesions), or disruptions. In particular, examples of retinal pathological elements may include characteristics and/or subtypes of the different types of elements and disruptions described above that can be detected and identified via retinal segmentation. For example, whether a retinal fluid is clear or turbid may be detectable and identifiable characteristic of the retinal fluid. Accordingly, in some examples, a retinal pathological element may be clear IRF, turbid IRF, clear SRF, turbid SRF, some other type of clear retinal fluid, some other type of turbid retinal fluid, or a combination thereof. In some cases, for SHRM, shape characteristics (e.g., tall SHRM, dome-shaped SHRM at the foveal center, flat SHRM near the foveal center, dysmorphic, etc.), boundary characteristics (e.g., ill-defined SHRM, well-defined SHRM), reflectivity (e.g., increased reflectivity or other levels of reflectivity), layering characteristics (e.g., hyperreflective bands in SHRM lesions), and lesion characteristics (e.g., the height, width, and/or area of SHRM lesions) may be examples of retinal pathological elements that may be detected and identified via retinal segmentation.

In other embodiments, the images 101 that are processed by image processor 108 may take the form of CT images and segmentation tool 118 may be used to segment bone (e.g., spine), lung tissue, or some other type of tissue. In some embodiments, the images 101 may be MRI images of the brain and segmentation tool 118 is used to segment one or more ventricles of the brain.

In this manner, image processor 108 may be used to process different types of images (e.g., medical images/medical imaging) to identify regions of pixels that correspond to anatomical structures or features (e.g., tissue, bone, organs, fluid layers, etc.) of interest. Further, image processor 108 may generate adjusted boundary definitions that take into account corrections received via user input. The adjustment data 126 generated by image processor 108 may be used in various scenarios including, for example, forming training input for the supervised and/or semi-supervised training/retraining of machine learning models.

Thus, the set of anchor points may be utilized to reassign the class labels of the segmented regions of a tissue. In this way, the present techniques may allow machine-learning model-based image segmentations or other image segmentations to be corrected or adjusted to improve accuracy Further, the corrected segmentations (e.g., adjusted images 126) may be used to retrain segmentation model 118 and/or other models and further improve the performance and accuracy of these models. This may further ensure that machine-learning model-based image segmentations or other image segmentations, as well as new pharmaceuticals or other treatments developed based thereon, can be trusted and made suitable for approval for clinical trials and usage by governmental or regulatory institutions.

### III. Example Methodologies of Image Analysis

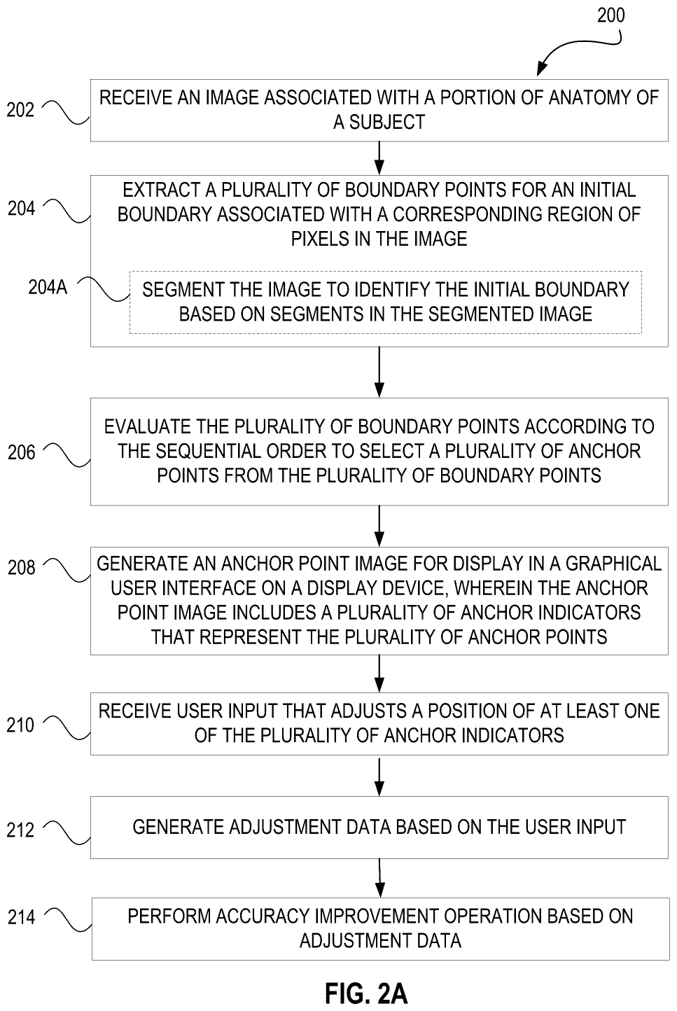

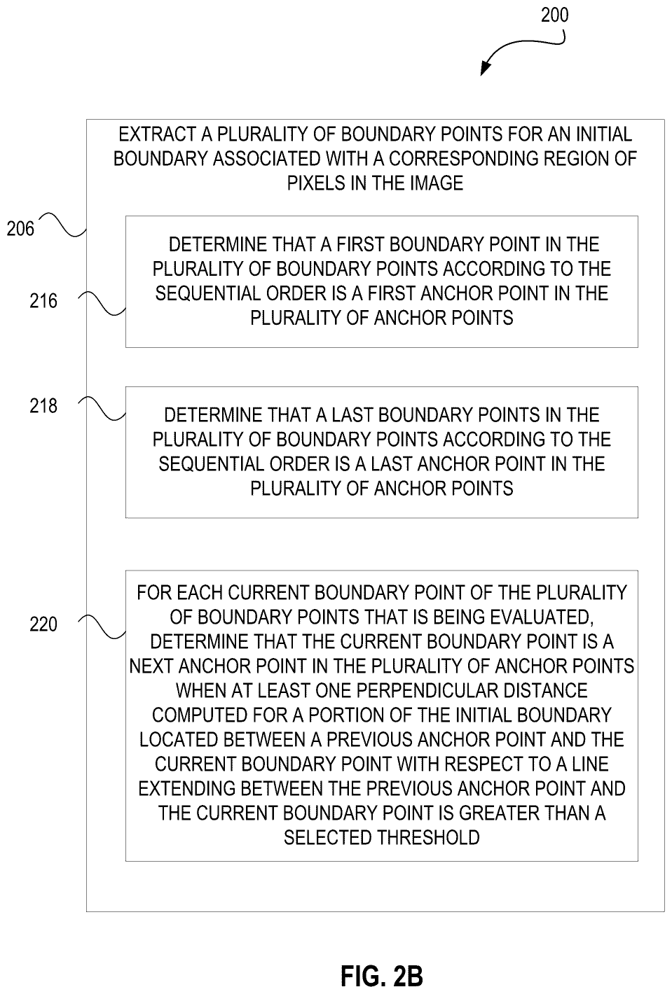

FIGS. 2A-2B form a flowchart of a process for generating adjustment data in accordance with one or more example embodiments.

FIG. 2A is a flowchart of a process 200 for generating adjustment data in accordance with one or more example embodiments. Process 200 may be implemented using, for example, without limitation, the image analysis system 100 described with respect to FIGS. 1A-1B. For example, process 200 includes various operations (steps) that may be performed using image processor 108 in FIGS. 1A-1B.

Process 200 includes step 202, which includes receiving an image associated with a portion of anatomy of a subject. The portion of anatomy may be, for example, without limitation, a retina of an eye, a brain, a lung, a heart, some other type of organ or anatomical structure, or a portion thereof. The image received in operation 202 may be a 2D image or a 3D image. In one or more embodiments, the image is an MRI image, a CT image, an X-ray image, an ultrasound image, a PET image, a SPECT image, an OCT image (e.g., an OCT volume, an OCT B-scan), or some other type of 2D/3D image that captures the portion of anatomy. The image received in step 202 may be, for example, image 114 or another one of images 101 described with respect to FIGS. 1A-1B.

In some embodiments, the image is a segmented image that has been generated from an initial image that is processed using a machine learning model (e.g., deep learning model). For example, the image received in step 202 may be segmented image 120 or another one of segmented images 116 described with respect to FIGS. 1A-1B. The segmented image may have been generated using, for example, a model (e.g., segmentation model 118 described with respect to FIGS. 1A-1B) performing artificial intelligence-based semantic segmentation.

In one or more embodiments, each group of pixels in the initial image is classified as belonging to one of a plurality of selected categories (e.g., selected categories 128 in FIG. 1B) and may form a “segment” in the segmented image. A segment may be a continuous or discontinuous grouping of pixels. For example, a segment may be formed by one or more separate regions of pixels, each of which is labeled as belonging to a same category. Alternatively, a segment may be an individual region of pixels such that a particular grouping of pixels may be represented in the segmented image by one or more segments. In this manner, the segmented image includes a plurality of regions of pixels (e.g., plurality of regions of pixels 130 in FIG. 1B).

Process 200 includes step 204, which includes extracting a plurality of boundary points for an initial boundary associated with a corresponding region of pixels in the image.

When the image received in step 202 is a segmented image, extracting the plurality of boundary points may include identifying the initial boundary associated with at least one segment in the segmented image and then identifying the pixels that form the initial boundary as boundary points. In some embodiments, extracting the plurality of boundary points includes identifying the pixels that form an initial boundary as the plurality of boundary points such that identification of the initial boundary and the plurality of boundary points occurs at the same time or at substantially the same time. In other embodiments, the segmented image may include a curved line that identifies the initial boundary such that step 204 includes identifying the pixels that form the initial boundary as boundary points.

When the image received in step 202 is an image that has not yet been segmented such as, for example, an OCT image, an MRI image, a CT image, an X-ray image, an ultrasound image, a PET image, an SPECT image, or some other type of image, process 200 may optionally include operation 204a, which includes segmenting the image to identify the initial boundary based on the segments in the segmented image.

The plurality of boundary points is associated with a sequential order. The sequential order may be defined with respect to a reference two-dimensional (2D) plane. When the image received in step 202 is a 2D image, the 2D plane may be the same X, Y plane of the image, which may be formed by the XY pixel coordinate system of the image itself. When the image is a 3D image, the sequential order may be defined with respect to a plurality of two-dimensional planes (or slices) that are parallel a reference 2D plane. The initial boundary described in step 204 may be, for example, initial boundary 136 in FIG. 1B. The plurality of boundary points described in step 204 may be, for example, boundary points 138 in FIG. 1B.

Process 200 includes step 206, which includes evaluating the plurality of boundary points according to the sequential order to select a plurality of anchor points from the plurality of boundary points. Evaluating the plurality of boundary points in step 206 may be performed via sub-operations (sub-steps). Example of sub-operations that may be performed as part of step 206 are described in further detail with respect to FIG. 2B below.

Process 200 further includes step 208, which includes generating an anchor point image for display in a graphical user interface on a display device, wherein the anchor point image includes a plurality of anchor indicators that represent the plurality of anchor points. The anchor point image may be, for example, anchor point image 142 that includes plurality of anchor indicators 144 described with respect to FIG. 1B.

In one or more embodiments, the anchor point image includes the image received in step 202, and the anchor indicators are overlaid on or otherwise displayed over this image. As previously discussed, this image may be the original image (e.g., OCT image, MRI image, CT image, etc.) from which a segmented image is then generated. The plurality of anchor indicators may be controllable, movable, or otherwise manipulatable graphical indicators (e.g., a graphical shape or icon that indicates that the corresponding position has been determined to be an anchor point).

Process 200 may optionally include step 210, which includes receiving user input that adjusts a position of at least one of the plurality of anchor indicators. For example, the user may enter user input that moves an anchor indicator to a different position that is more accurately located on the actual boundary of interest. The movement of an anchor indicator causes a change from an original position of the corresponding anchor point to a final position that is different.

Process 200 may optionally include step 212, which includes generating adjustment data based on the user input. The adjustment data may be, for example, adjustment data 124 in FIGS. 1A-1B. The adjustment data can be used to generate labeled images that may be used to train or retrain machine learning models. For example, the labeled images may be used to train or retrain a segmentation model (e.g., segmentation model 118) used to generate the segmented image described above. In one or more embodiments, the adjustment data includes an adjusted version of the segmented image in which one or more pixels in the segmented image are reclassified from a first category (or label) to a second category (or label) based on the final positions of the anchor points. This adjusted version of the segmentation image (e.g., adjusted segmented image) can be used to form training input for the segmentation model or a different model. Using the adjusted version of the segmentation image for training may improve the accuracy and efficiency of the segmentation model in classifying pixels.

Process 200 may optionally include step 214, which includes performing an accuracy improvement operation based on the adjustment data. The accuracy improvement operation may include any number of operations itself. The accuracy improvement operation may include generated adjusted images that can be labeled images for use in retraining a segmentation model as described above. If the adjustment data includes labeled images, the accuracy improvement operation may include retraining the segmentation model based on the labeled images to improve performance of the segmentation model. The accuracy improvement operation may include using the adjustment data as or to form input images for another algorithm or model to improve the accuracy of model output as compared to using images 101 or segmented images 116 as input.

FIG. 2B is a more detailed flowchart of step 206 of the process 200 in accordance with one or more example embodiments. Performing step 206 may include performing various sub-operations including, for example, operation 216, operation 218, and operation 220.

Operation 216 includes determining that a first boundary point in the plurality of boundary points according to the sequential order is a first anchor point in the plurality of anchor points.

Operation 218 includes determining that a last boundary points in the plurality of boundary points according to the sequential order is a last anchor point in the plurality of anchor points. In some embodiments, operation 216 and operation 218 are performed as part of the same step.

Operation 220 includes, for each current boundary point of the plurality of boundary points that is being evaluated, determining that the current boundary point is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold. Accordingly, each boundary point in the sequential order may be evaluated for inclusion in the plurality of anchor points based on the one or more boundary points that come before the current boundary point in the sequential order being evaluated and after the previous anchor point. The current boundary point may be also referred to as a check point.

In operation 220, for example, the one or more boundary points located between the previous anchor point and the current boundary point may be referred to as a set of middle boundary points. Operation 220 may include computing a perpendicular distance from each middle boundary point in the set of middle boundary points to the line extending between the previous anchor point and the current boundary point to form a set of perpendicular distances. If at least one of these perpendicular distances is greater than the selected threshold, the current boundary point (i.e., check point) is determined to be the next anchor point. The selected threshold may be, for example, a distance that is between 1-15 in the units of the reference 2D plane. The units may be pixel units.

The selected threshold may be selected to control the density of anchor points that are selected from the boundary points. In other words, the threshold may be selected to control the complexity of curvature, grooves, texture, and/or contour in the boundary that is represented by the anchor points. A higher threshold may be selected to reduce the density (number of) anchor points and reduce the complexity of curvature, grooves, texture, and/or contour in the boundary that is captured. In contrast, a lower threshold may be selected to increase the density (number of) anchor points and increase the complexity of curvature, grooves, texture, and/or contour in the boundary that is captured.

If there are no middle boundary points located between the current boundary point being evaluated and the previous anchor point and the current boundary point is not the last boundary point, the next boundary point is then evaluated.

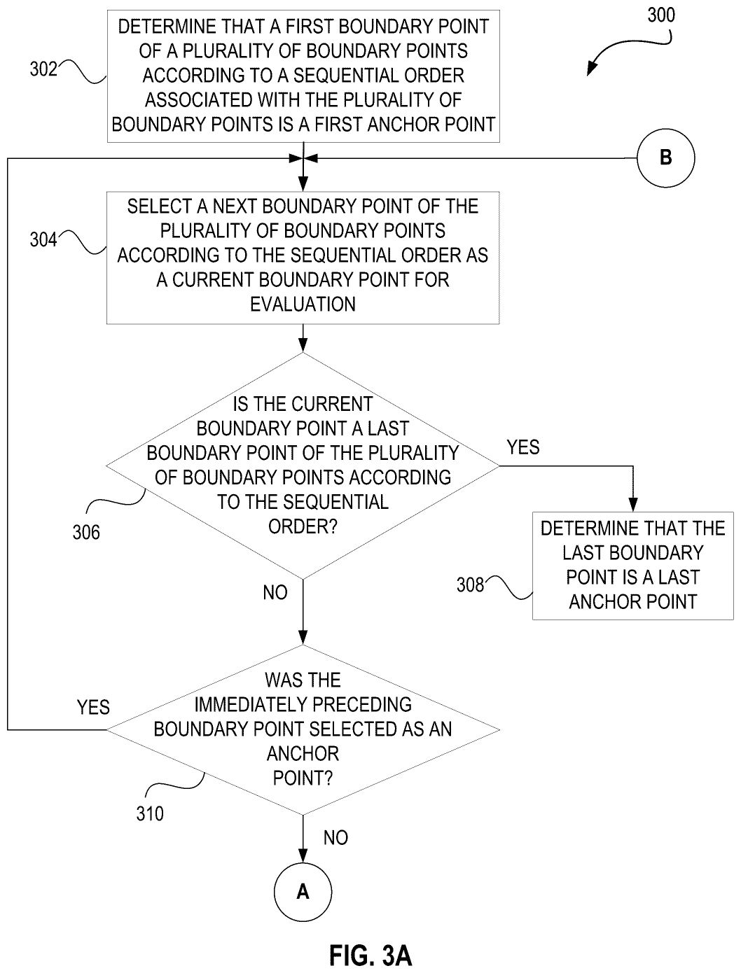

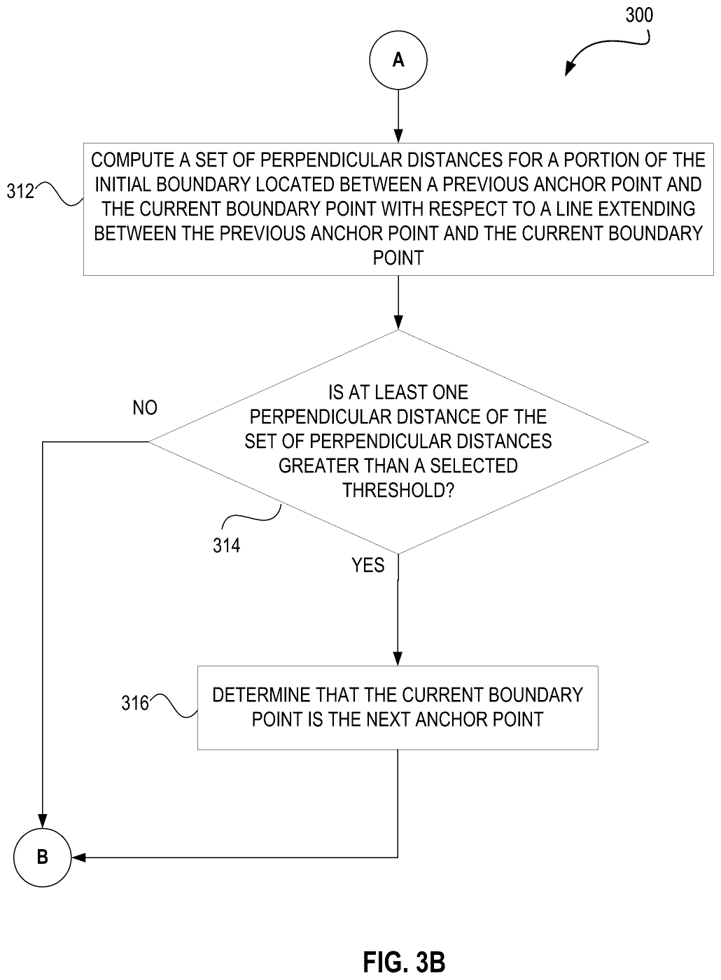

FIGS. 3A-3B together are flowchart of a process for evaluating boundary points in accordance with one or more example embodiments. Process 300 may be implemented using, for example, without limitation, the image analysis system 100 described with respect to FIGS. 1A-1B. For example, process 300 includes various operations (steps) that may be performed using image processor 108 in FIGS. 1A-1B. Process 300 may be one example of implementing step 206 in FIGS. 2A-2B. Process 300 is specific to a selected 2D plane of boundary points being evaluated and may be performed for each 2D plane of boundary points being evaluated.

Step 302 includes determining that a first boundary point of a plurality of boundary points according to a sequential order associated with the plurality of boundary points is a first anchor point.

Step 304 includes selecting a next boundary point of the plurality of boundary points according to the sequential order as a current boundary point for evaluation.

Step 306 includes determining whether the current boundary point is a last boundary point of the plurality of boundary points according to the sequential order. If the current boundary point is the last boundary point of the plurality of boundary points according to the sequential order, process 300 performs step 308.

Step 308 includes determining that the last boundary point is a last anchor point. This is the last boundary point for the initial boundary. Process 300 then terminates.

With reference again to step 308, if the current boundary point is not the last boundary point of the plurality of boundary points according to the sequential order, process 300 performs step 310.

Step 310 includes determining whether the immediately preceding boundary point was selected as an anchor point. If the immediately preceding boundary point was selected as an anchor point, process 300 returns to step 304 described above. If, however, the immediately preceding boundary point was not selected as an anchor point, process 300 then performs step 312.

Step 312 includes computing a set of perpendicular distances for a portion of the initial boundary located between a previous anchor point and the current boundary point with respect to a line extending between the previous anchor point and the current boundary point. For example, the plurality of boundary points for the initial boundary may include one or more boundary points between the previous anchor point and the current boundary point. This one or more boundary points may be referred to as the “set of middle boundary points.” A perpendicular distance is computed for each middle boundary point in this set of middle boundary points.

Step 314 includes determining whether at least one perpendicular distance of the set of perpendicular distances that is computed is greater than a selected threshold. The selected threshold may be, for example, a threshold selected between 1 and 10, where the threshold is in pixel units. If no perpendicular distance in the set of perpendicular distances is greater than the selected threshold, process 300 returns to step 304 described above.

The threshold may be selected to control the density of anchor points that are selected from the boundary points. In other words, the threshold may be selected to control the complexity of curvature, grooves, texture, and/or contour in the boundary that is represented by the anchor points. A higher threshold may be selected to reduce the density (number of) anchor points and reduce the complexity of curvature, grooves, texture, and/or contour in the boundary that is captured. In contrast, a lower threshold may be selected to increase the density (number of) anchor points and increase the complexity of curvature, grooves, texture, and/or contour in the boundary that is captured.

With reference again to step 314, if at least one perpendicular distance of the set of perpendicular distances is greater than the selected threshold, process 300 performs step 316.

Step 316 includes determining that the current boundary point is the next anchor point, with process 300 then returning to operation 304 as described above.

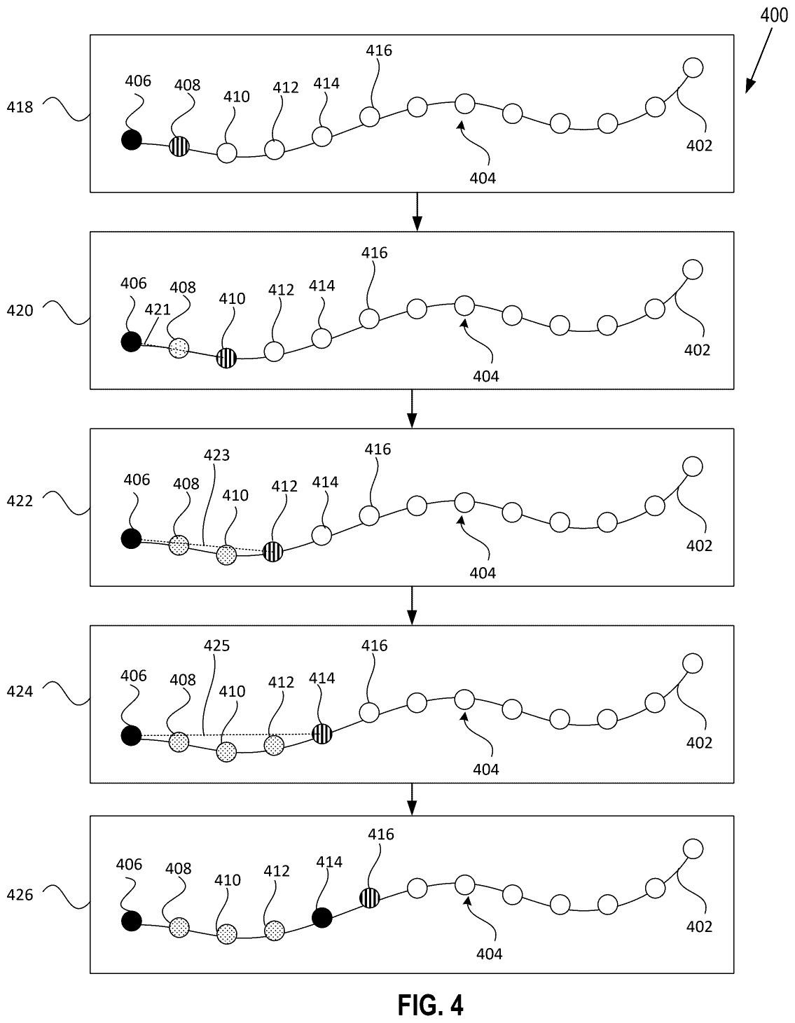

FIG. 4 is a workflow diagram of a process for evaluating a plurality of boundary points for possible inclusion in a plurality of anchor points in accordance with one or more embodiments. Workflow 400 may be one example of an implementation for step 206 in FIG. 2. Workflow 400 may be implemented using image processor 108 in FIGS. 1A-1B. For example, workflow 400 may be implemented using adjustment tool 112 of image processor 108 in FIGS. 1A-1B. Workflow 400 illustrates one manner in which at least a portion of the process 300 described with respect to FIGS. 3A-3B may be implemented.

Initial boundary 402 is one example of an implementation for initial boundary 136 in FIG. 1B. Initial boundary 402 may be a boundary identified from a segmented image (e.g., segmented image 120 in FIG. 1). Image processor 108 extracts plurality of boundary points 404 from initial boundary 402. Plurality of boundary points 404 may be one example of an implementation for plurality of boundary points 138 in FIG. 1B.

Plurality of boundary points 404 include, for example, boundary points 406, 408, 410, 412, 414, and 416 as well as other points along initial boundary 402 in FIG. 4. Each of the boundary points in plurality of boundary points 404 corresponds to a pixel of the segmented image and has an X,Y position (e.g., in pixel units). Plurality of boundary points 404 are evaluated in a sequential order (which is shown as an order form left to right in FIG. 4). In workflow 400, anchor points are depicted using black solid fill, middle boundary points are depicted using a dotted pattern, and the current boundary being evaluated (i.e., the check point) at a given step is depicted suing a vertical stripe pattern.

Because boundary point 306 is the first boundary point in the sequential order from left to right, boundary point 406 is determined to be the first anchor point, and a next boundary point is selected for evaluation.

At step 418, boundary point 408 is the next to be evaluated. Because boundary point 406, which immediately precedes boundary point 408, is a previous anchor point (e.g., the first anchor point), a next boundary point is selected for evaluation.

At step 420, boundary point 410 is the next to be evaluated. Boundary point 408 is now a middle boundary point between the previous anchor point (boundary point 406) and the current boundary point (boundary point 410). Line 421 extends between previous anchor point (boundary point 406) and the current boundary point (boundary point 410). A determination is made as to whether a perpendicular distance from boundary point 408 to line 421 is greater than the selected threshold (e.g., 1, 1.25, 1.5, 2, 2.5, 3, 3, 5, 4, 5, 6, 7, 8, 9, 10, etc. or some other number of pixel units between 1 and 15). Because here, the computed perpendicular distance is not greater than the threshold, a next boundary point is selected for evaluation.

At step 422, boundary point 412 is evaluated. Boundary points 408 and 410 are now the middle boundary points between the previous anchor point (boundary point 406) and the current boundary point (boundary point 412,). Line 423 extends between previous anchor point (boundary point 406) and the current boundary point (boundary point 412). The perpendicular distance from boundary point 408 to line 423 is computed. The perpendicular distance from boundary point 410 to line 423 is computed. A determination is made as to whether any one of these perpendicular distances is greater than the selected threshold. Because here, none of the computed perpendicular distances is greater than the selected threshold, a next boundary point is selected for evaluation.

At step 424, boundary point 414 is evaluated. Boundary points 408, 410, and 412 are now the middle boundary points between the previous anchor point (boundary point 406) and the current boundary point (boundary point 414,). Line 425 extends between previous anchor point (boundary point 406) and the current boundary point (boundary point 414). The perpendicular distance from boundary point 408 to line 425 is computed. The perpendicular distance from boundary point 410 to line 425 is computed. The perpendicular distance from boundary point 412 to line 425 is computed. A determination is made as to whether at least one of these perpendicular distances is greater than the selected threshold. Because here, the computed perpendicular distance from boundary point 410 to line 425 is determined to be greater than the threshold, the current boundary point (boundary point 414) is determined to be the next anchor point, and a next boundary point is selected for evaluation.

At step 426, boundary point 416 is evaluated. At step 426, boundary point 414 is now the previously identified anchor point. At step 426, because boundary point 414, which immediately precedes boundary point 416, is the previous anchor point, a next boundary point is selected for evaluation.

The above-described process of evaluation is completed for each of the remaining boundary points with the last boundary point in the plurality of boundary points being selected as the last (or final) anchor point for initial boundary 402.

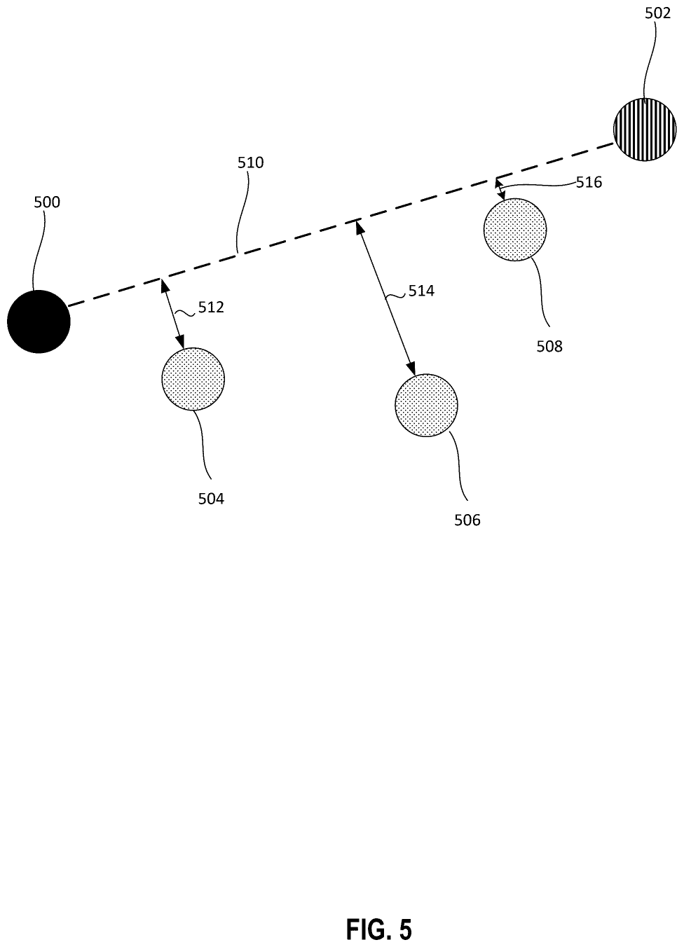

FIG. 5 is a diagram illustrating how a perpendicular distance is computed in accordance with one or more example embodiments. In FIG. 5, previous anchor point 500 is a previously identified anchor point such as, for example, without limitation, boundary point 406 in FIG. 4. Current boundary point 502 (which may also be referred to as a check point) is the current boundary point being evaluated. Set of middle boundary points 503 may include all boundary points between previous anchor point 500 and current boundary point 502. Here, set of middle boundary points 503 includes middle boundary point 504, middle boundary point 506, and middle boundary point 508.

Line 510 may be a line (e.g., theoretical/abstract/imaginary/virtual/computed) line extending between previous anchor point 500 and current boundary point 502. A perpendicular distance is computed for each middle boundary point of set of middle boundary points 503. A perpendicular distance may be measured as the length of a line that extends perpendicularly between the position of a point of interest (e.g., a middle boundary point) to a reference line (e.g., the line extending between the previous anchor point and the current boundary point).

Here, perpendicular distance 512, perpendicular distance 514, and perpendicular distance 516 are computed for boundary point 504, boundary point 506, and boundary point 508, respectively, with respect to line 510. If any one of these perpendicular distances is determined to be greater than a selected threshold, then current boundary point 502 is determined to be the next anchor point.

The threshold may be selected to control the density of anchor points that are selected from the boundary points. In other words, the threshold may be selected to control the complexity of curvature, grooves, texture, and/or contour in the boundary that is represented by the anchor points. A higher threshold may be selected to reduce the density (number of) anchor points and reduce the complexity of curvature, grooves, texture, and/or contour in the boundary that is captured. In contrast, a lower threshold may be selected to increase the density (number of) anchor points and increase the complexity of curvature, grooves, texture, and/or contour in the boundary that is captured.

Using anchor points and anchor indicators that represent those anchor points in the manner described above with respect to FIGS. 2A, 2B, 3A, 3B, 4, and 5 to enable a user to correct the boundaries identified in segmented images reduces the overall time and resources needed to adjust/correct segmented images to form adjusted images. Because the anchor indicators represent only a subset of the boundary points extracted for a given boundary, adjustments to the overall boundary may be more efficiently made without the user needing to adjust each boundary point (pixel) of that boundary. Further, with fewer anchor indicators, there are fewer overall adjustment computations that need to be made without sacrificing overall accuracy. Thus, generating anchor points and a graphical user interface that displays controllable/movable anchor indicators that represent the anchor points may improve the overall functioning of the image analysis system performing the methods described herein and may reduce overall consumption of computing resources.

### IV. Examples of Graphical User Interface Displays

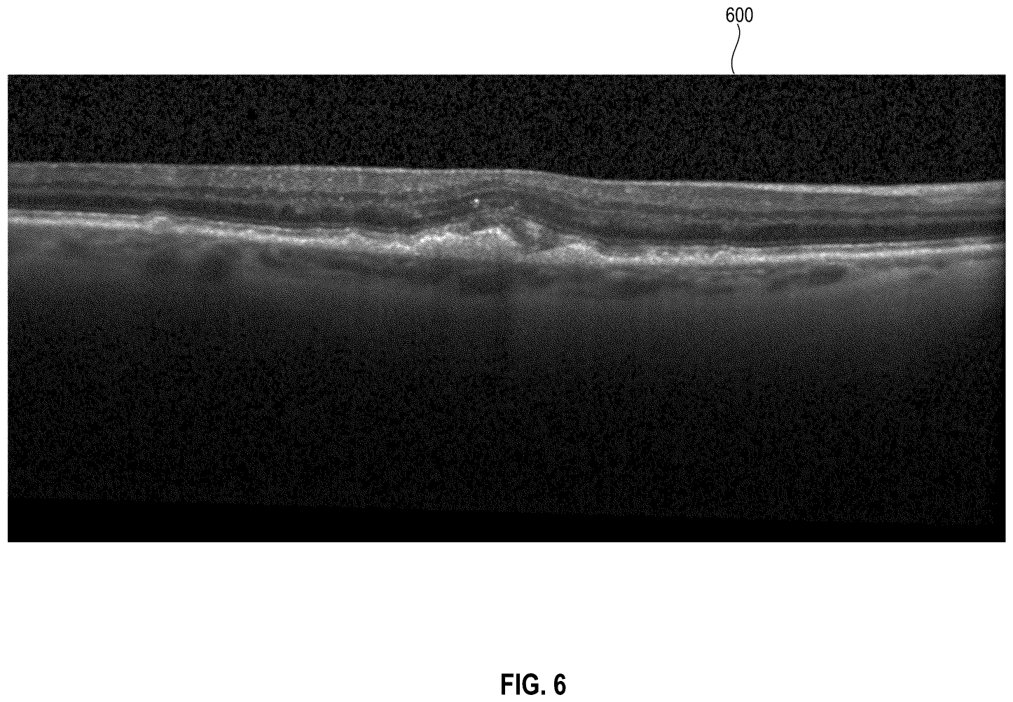

FIGS. 6-16 are illustrations showing the types of images that may be displayed on a graphical user interface in accordance with one or more example embodiments. The graphical user interface may be, for example, graphical user interface 122 in FIGS. 1A-1B. The different images shown in FIGS. 6-16 show how an image (e.g., OCT B-scan) can be processed using the image analysis system 100 described with respect to FIGS. 1A-1B and the methodologies described with respect to FIGS. 2A, 2B, 3A, 3B, 4, and 5 to identify boundaries of interest, corresponding boundary points, and corresponding anchor points. These anchor points may be displayed on the image using anchor indicators that are manipulable by a user so that the user can make adjustments to more accurately identify boundaries of interest.

FIG. 6 is an illustration of an image displayed in a graphical user interface in accordance with one or more example embodiments. Image 600 is one example of an implementation for image 114 described with respect to FIGS. 1A-1B. Image 600 may be a retinal image that captures the retina of a subject. For example, image 600 may be an OCT-B scan.

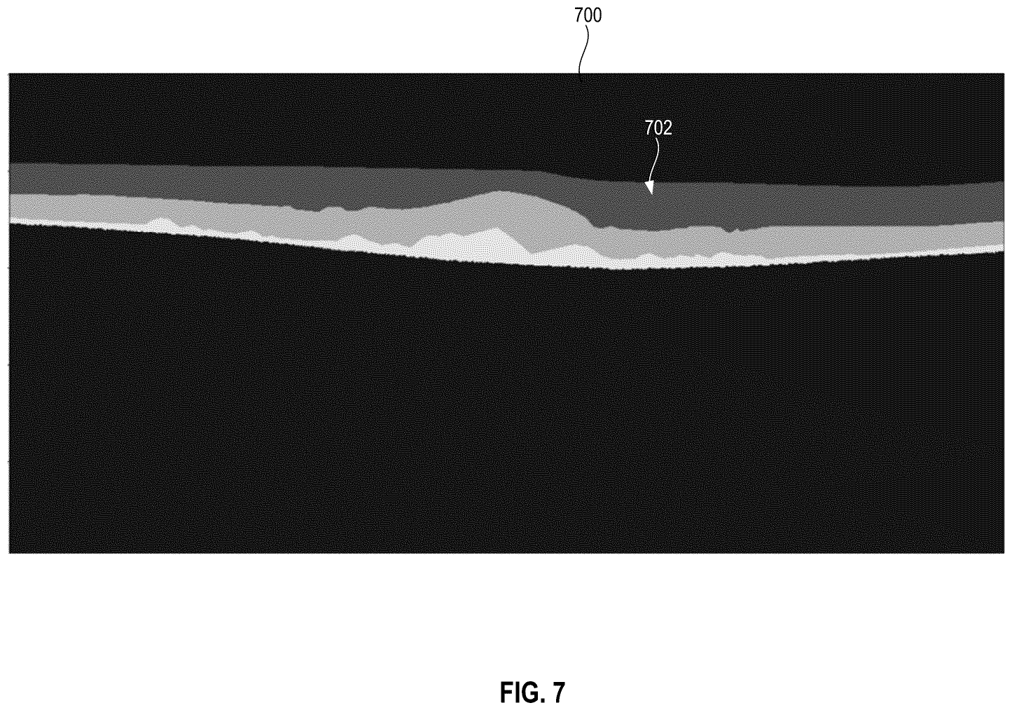

FIG. 7 is an illustration of a segmented image displayed in a graphical user interface in accordance with one or more example embodiments. Segmented image 700 is one example of an implementation for segmented image 120 described with respect to FIGS. 1A-1B. Segmented image 700 may have been generated using, for example, segmentation model 118 of segmentation tool 110 in image processor 108 in FIGS. 1A-1B. Segmented image 700 identifies a plurality of regions of pixels 702. In this example, each region of pixels is a “segment” that corresponds to a different retinal element. For example, image 600 in FIG. 6 may be segmented to detect and identify one or more retinal (e.g., retina-associated) elements. A retinal element may be comprised of at least one of a retinal layer element or a retinal pathological element.

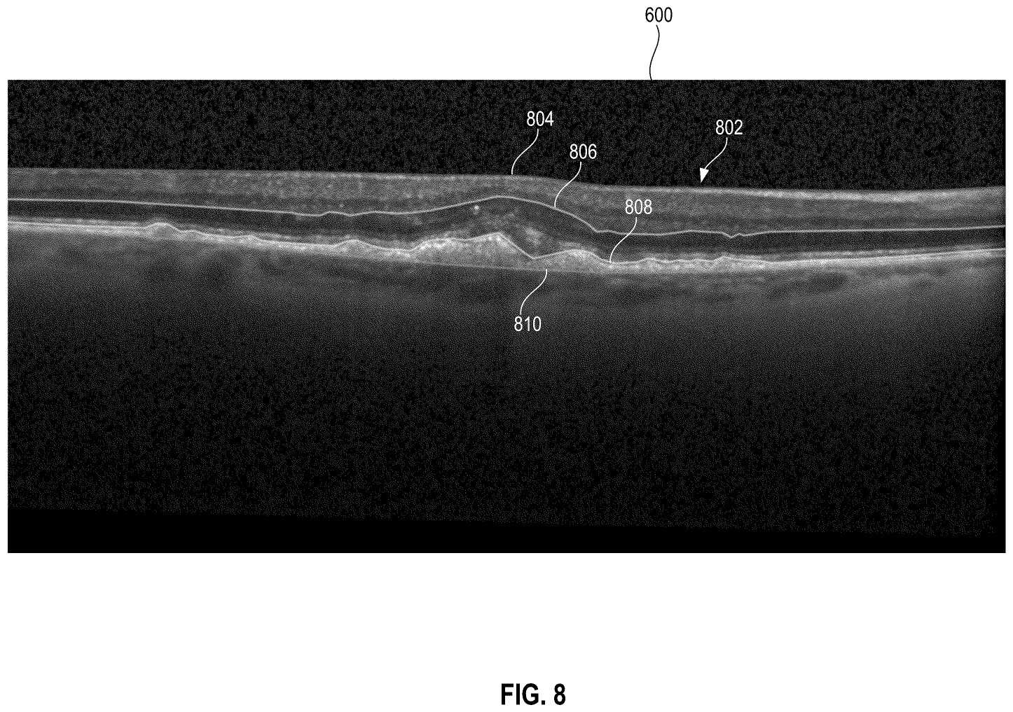

FIG. 8 is an illustration of an image with identified boundaries displayed in a graphical user interface in accordance with one or more example embodiments. Image 600 from FIG. 6 is displayed with a set of initial boundaries 802 overlaid on image 600. Set of initial boundaries 802 include initial boundary 804, initial boundary 806, initial boundary 808, and initial boundary 810. Set of initial boundaries 802 is one example of an implementation for set of initial boundaries 134 in FIG. 1B.

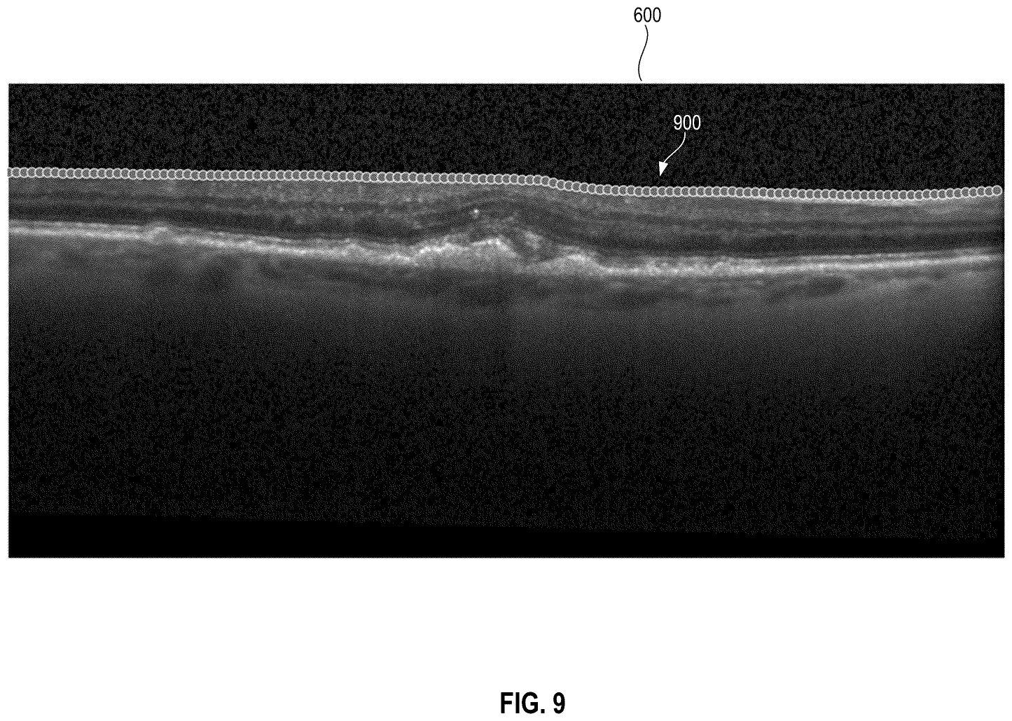

FIG. 9 is an illustration of an image displayed with boundary indicators in a graphical user interface in accordance with one or more example embodiments. Image 600 from FIG. 6 is displayed with boundary indicators 900 that represent corresponding boundary points that have been extracted for initial boundary 804 from FIG. 8. The boundary points represented by boundary indicators 900 may be one example of an implementation for boundary points 138 in FIG. 1B.

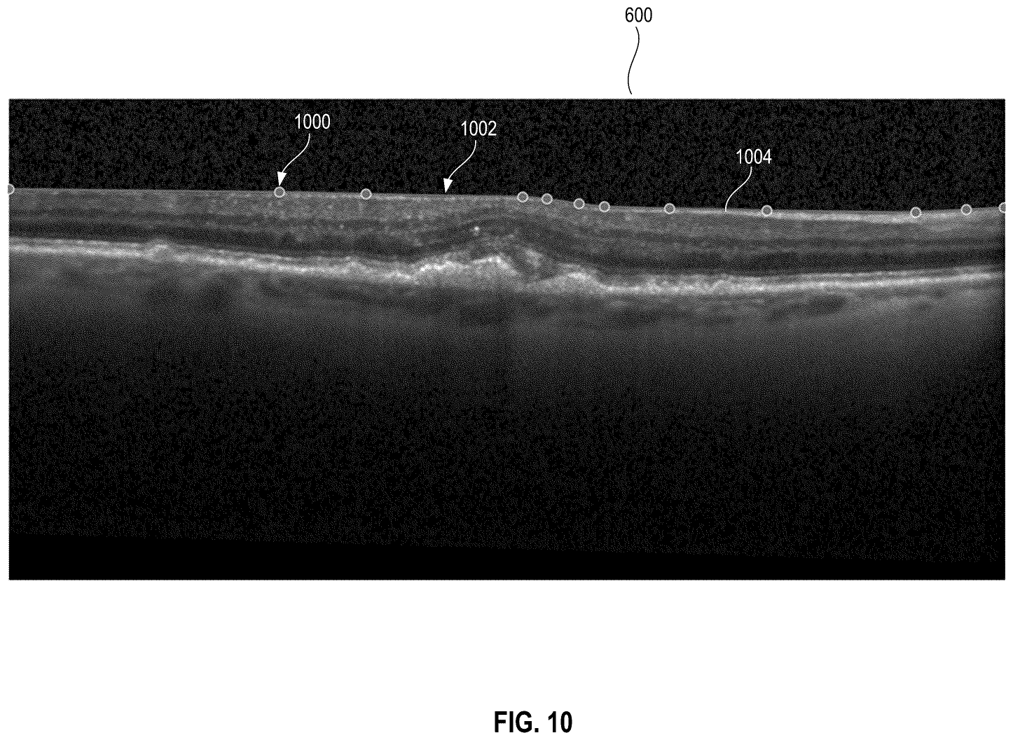

FIG. 10 is an illustration of an image with anchor indicators displayed in a graphical user interface in accordance with one or more example embodiments. Image 600 from FIG. 6 is displayed with anchor indicators 1000 that represent anchor points that have been determined for initial boundary 804 from FIG. 8. Anchor indicators 1000 may be one example of an implementation for anchor indicators 144 in FIG. 1B. Anchor indicators 1000 are connected by line segments 1002 that form new boundary 1004. New boundary 1004 approximates initial boundary 804 from FIG. 8, reducing the complexity of curvature, grooves, texture, and/or contour in initial boundary 804 within selected tolerances.

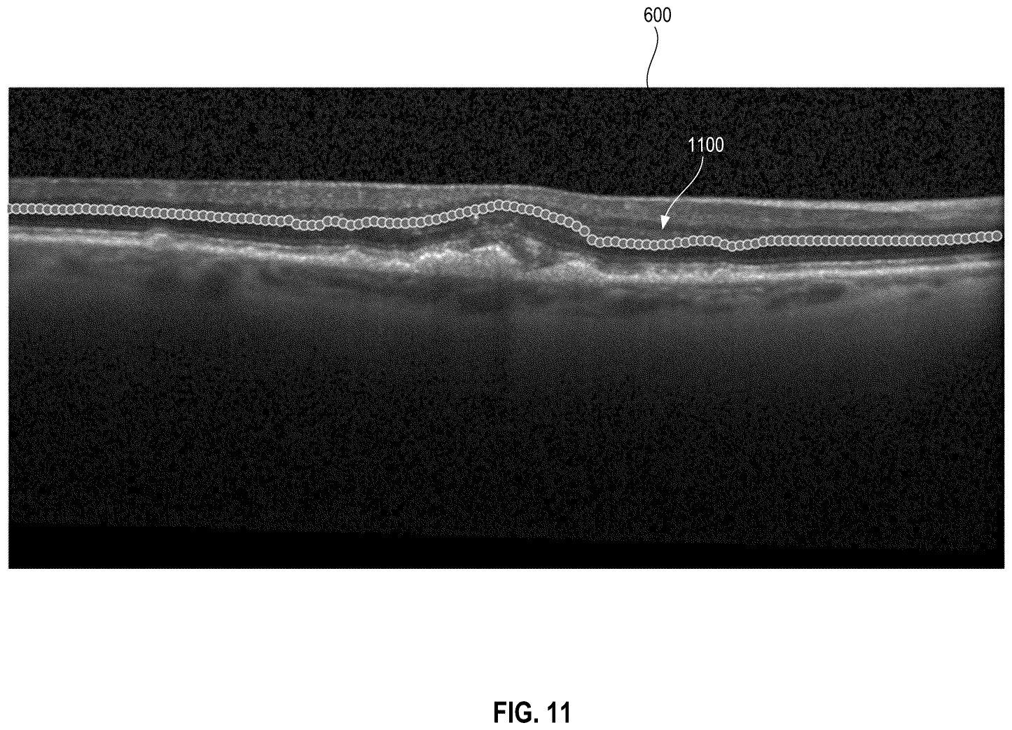

FIG. 11 is an illustration of an image displayed with boundary indicators in a graphical user interface in accordance with one or more example embodiments. Image 600 from FIG. 6 is displayed with boundary indicators 1100 that represent boundary points that been extracted for initial boundary 806 from FIG. 8. The boundary points represented by boundary indicators 1100 may be one example of an implementation for boundary points 138 in FIG. 1B.

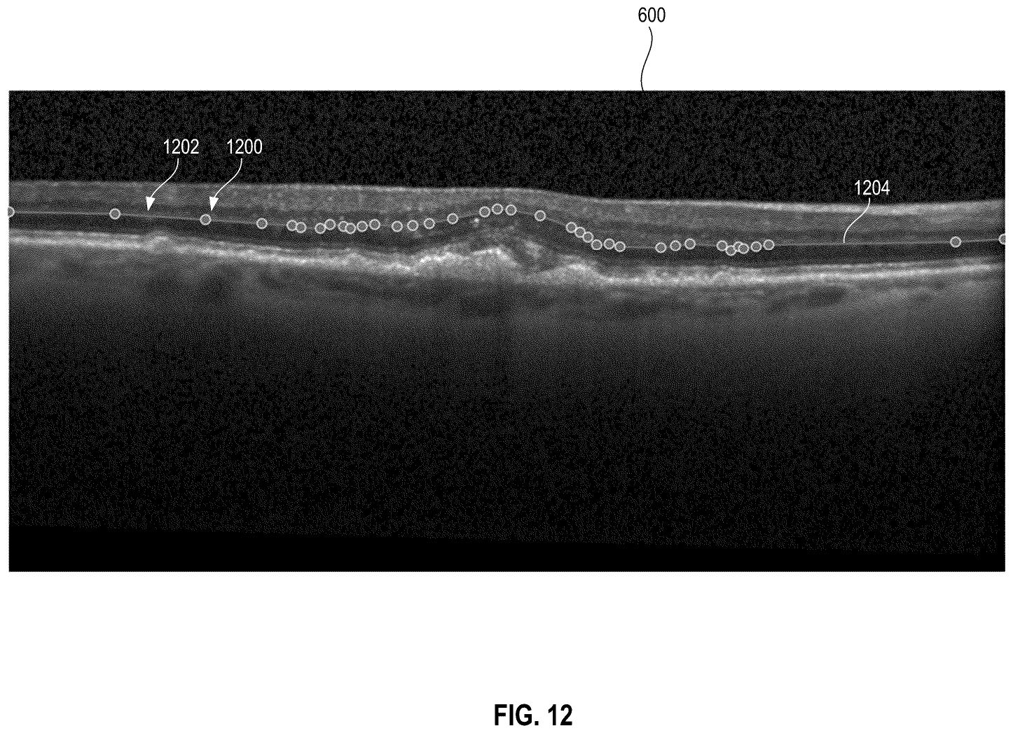

FIG. 12 is an illustration of an image with anchor indicators displayed in a graphical user interface in accordance with one or more example embodiments. Image 600 from FIG. 6 is displayed with anchor indicators 1200 that represent anchor points have been determined for initial boundary 806 from FIG. 8. Anchor indicators 1200 may be one example of an implementation for anchor indicators 144 in FIG. 1B. Anchor indicators 1200 are connected by line segments 1202 that form new boundary 1204. New boundary 1204 approximates initial boundary 806 from FIG. 8, reducing the complexity of curvature, grooves, texture, and/or contour in initial boundary 804 within selected tolerances.

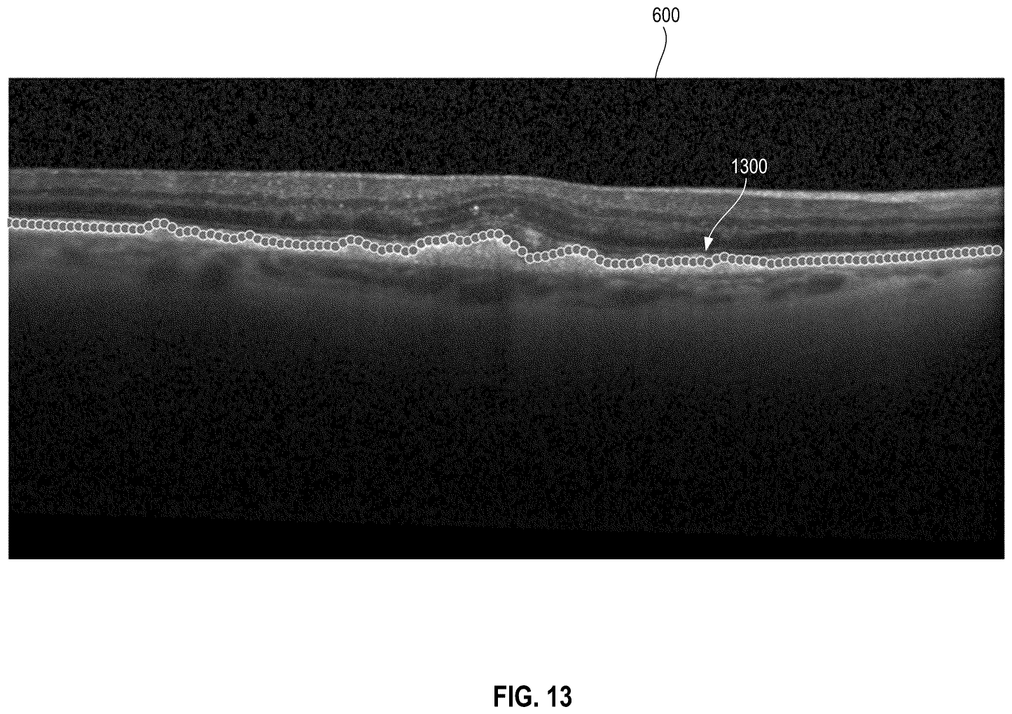

FIG. 13 is an illustration of an image displayed with boundary indicators in a graphical user interface in accordance with one or more example embodiments. Image 600 from FIG. 6 is displayed with boundary indicators 1300 that represent boundary points that have been extracted for initial boundary 808 from FIG. 8. The boundary points represented by boundary indicators 1300 may be one example of an implementation for boundary points 138 in FIG. 1B.

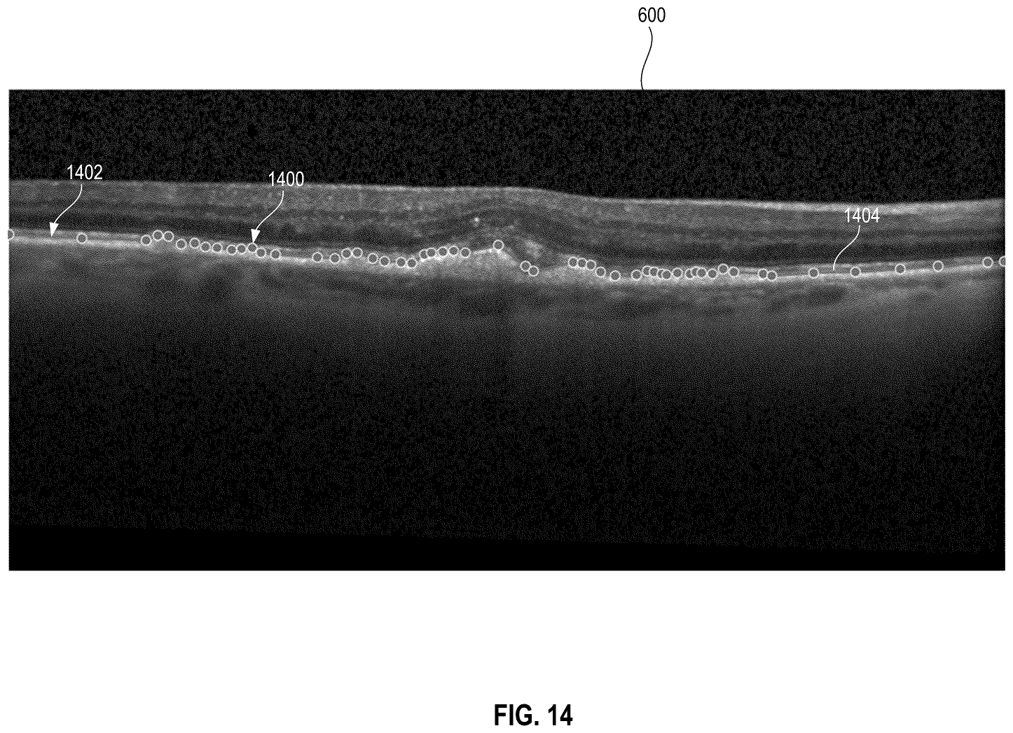

FIG. 14 is an illustration of an image with anchor points displayed in a graphical user interface in accordance with one or more example embodiments. Image 600 from FIG. 6 is displayed with anchor indicators 1400 that represent anchor points have been determined for initial boundary 808 from FIG. 8. Anchor indicators 1400 may be one example of an implementation for anchor indicators 144 in FIG. 1B. Anchor indicators 1400 are connected by line segments 1402 that form new boundary 1404. New boundary 1404 approximates initial boundary 808 from FIG. 8, reducing the complexity of curvature, grooves, texture, and/or contour in initial boundary 804 within selected tolerances.

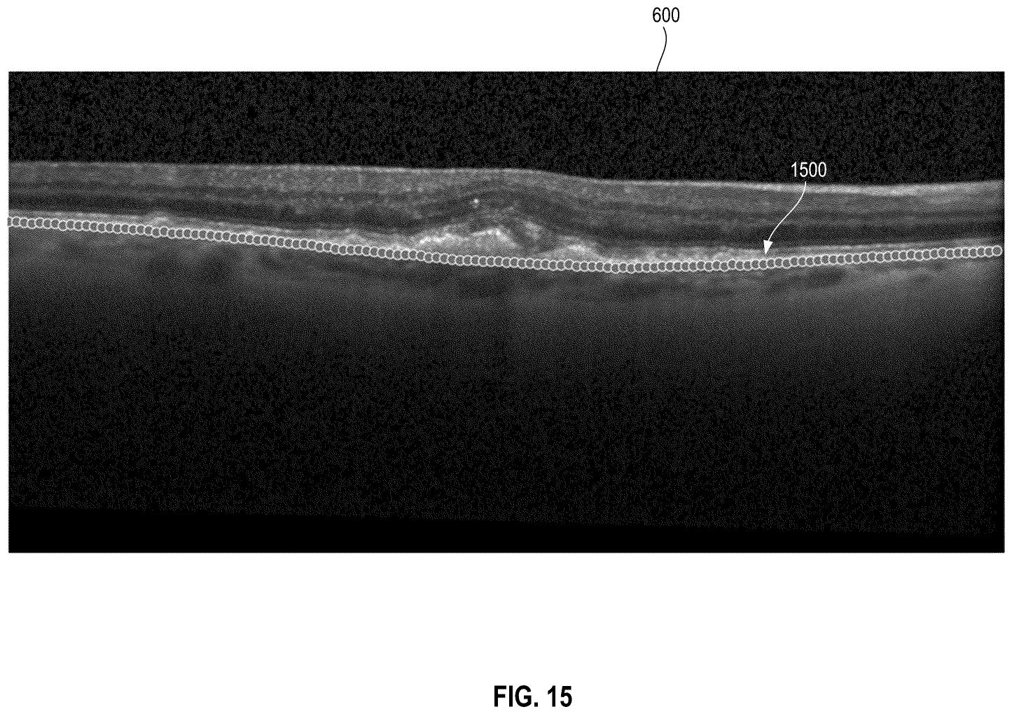

FIG. 15 is an illustration of an image displayed with boundary indicators in a graphical user interface in accordance with one or more example embodiments. Image 600 from FIG. 6 is displayed with boundary indicators 1500 that represent boundary points that have been extracted for initial boundary 810 from FIG. 8. The boundary points represented by boundary indicators 1500 may be one example of an implementation for boundary points 138 in FIG. 1B.

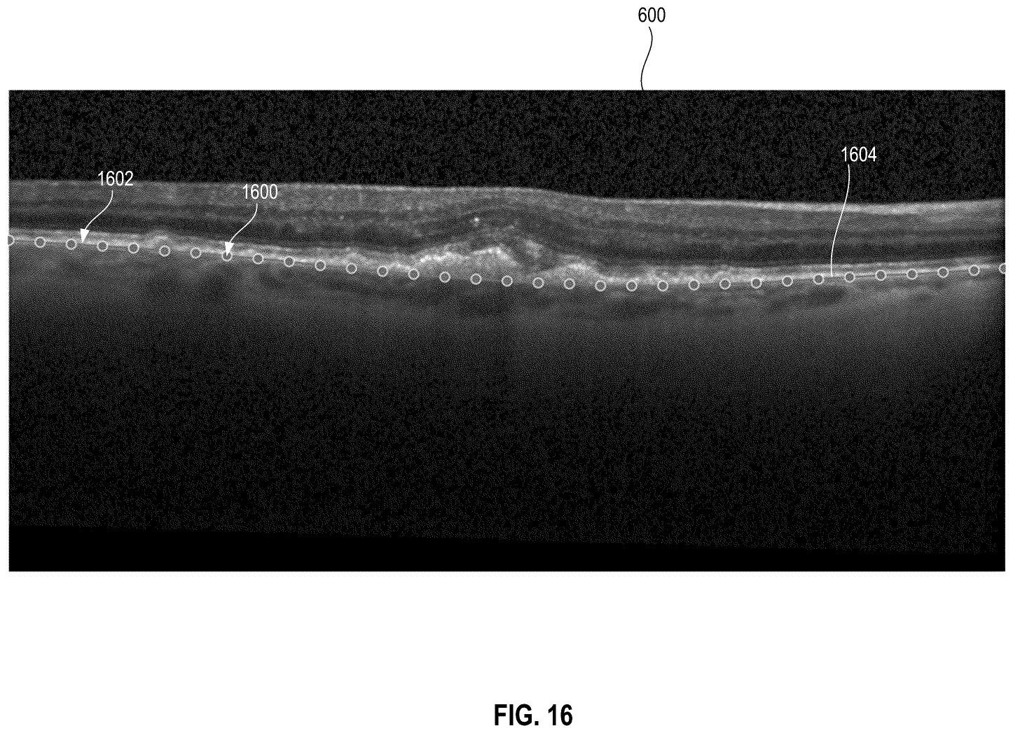

FIG. 16 is an illustration of an image with anchor points displayed in a graphical user interface in accordance with one or more example embodiments. Image 600 from FIG. 6 is displayed with anchor indicators 1600 that represent anchor points have been determined for initial boundary 810 from FIG. 8. Anchor indicators 1600 may be one example of an implementation for anchor indicators 144 in FIG. 1B. Anchor indicators 1600 are connected by line segments 1602 that form new boundary 1604. New boundary 1604 approximates initial boundary 810 from FIG. 8, reducing the complexity of curvature, grooves, texture, and/or contour in initial boundary 804 within selected tolerances.

As previously noted, the automatic selection of anchor points and display of corresponding anchor indicators such as those shown in FIGS. 10, 12, 14, and 16 allow a user (e.g., a medical expert, a medical professional, a human pathologist, a human grader, a reading center, etc.) to easily provide user input that can be used to then adjust, automatically, the previously identified boundaries to form new boundaries that more accurately represent the boundaries of interest.

Using anchor points and anchor indicators that represent those anchor points in the manner described above with respect to FIGS. 6-16 may enable adjustment/correction of segmented image 700 in FIG. 7 in a manner that reduces the overall time and resources needed to make these adjustments/corrections, without sacrificing accuracy. Because the anchor indicators represent only a subset of the boundary points extracted for a given boundary, adjustments to the overall boundary may be more efficiently made without the user needing to adjust each boundary point (pixel) of that boundary. Further, with fewer anchor indicators, there are fewer overall adjustment computations that need to be made without sacrificing overall accuracy. Thus, generating anchor points and a graphical user interface that displays controllable/movable anchor indicators that represent the anchor points may improve the overall functioning of the image analysis system described herein and may reduce overall consumption of computing resources.

Further, the image processor may generate adjustment data (e.g., adjustment images 126 as described with respect to FIGS. 1A-1B) that include or can be used to generate new segmented images in which the classification of pixels more accurately reflects the subject's actual anatomy/pathology. In some cases, the new segmented images (the adjusted images) may be used to better train and/or retrain machine learning models to improve segmentation performance. This type of improvement may help ensure that machine-learning model-based image segmentation and/or other image segmentation, as well as new pharmaceuticals or other treatments developed based thereon, can be trusted and made suitable for approval for clinical trials and usage by governmental or regulatory institutions.

For example, the segmentation of the layers of the retina may be used to determine whether lesions are present in the retina and to track changes in the one or more lesions over time. Thus, delineating accurate boundaries for each region or layer of the retina allows for proper identification and tracking of lesions in the retina. In certain embodiments, the set of anchor points may be configured to adjust the boundaries to accurately label the one or more lesions of the retina.

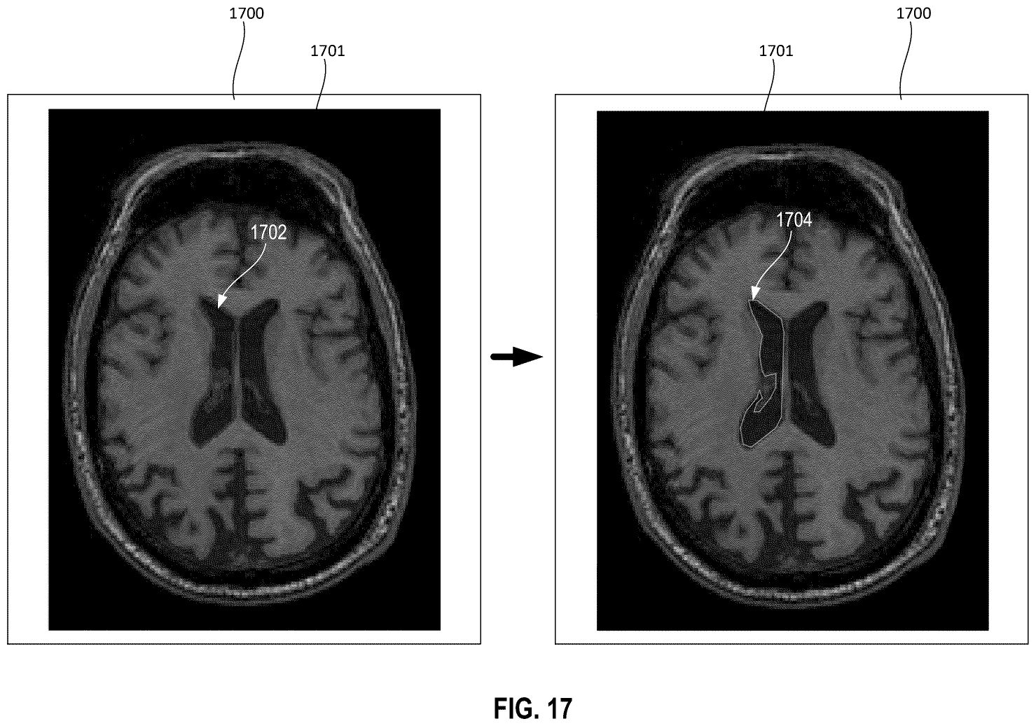

FIGS. 17-20 are illustrations of various displays that may be presented in a graphical user interface in accordance with one or more example embodiments. Graphical user interface 1700 is an example of one implementation for graphical user interface 122 in FIGS. 1A-1B.

FIG. 17 is an illustration of a graphical user interface transforming the display of a boundary to anchor indicators in accordance with one or more example embodiments. On the left, graphical user interface 1700 displays image 1701. Image 1701 is a high-resolution image (e.g., an MRI scan of a subject's brain. Image 1701 is displayed with boundary 1702 that identifies a ventricle of the brain.

The image processor 108 described in FIGS. 1A-1B may be used to transform the display of boundary 1702 to a display of anchor indicators 1704 over image 1701, as shown on the right. The anchor indicators 1704, when connected via line segments as shown, approximate boundary 1702 within selected tolerances. Anchor indicators 1704, which may be one example of an implementation for anchor indicators 144 in FIG. 1B, represent anchor points. Each of anchor indicators 1704 is a movable graphical indicator that can be moved via user input to adjust the position of a corresponding anchor point.

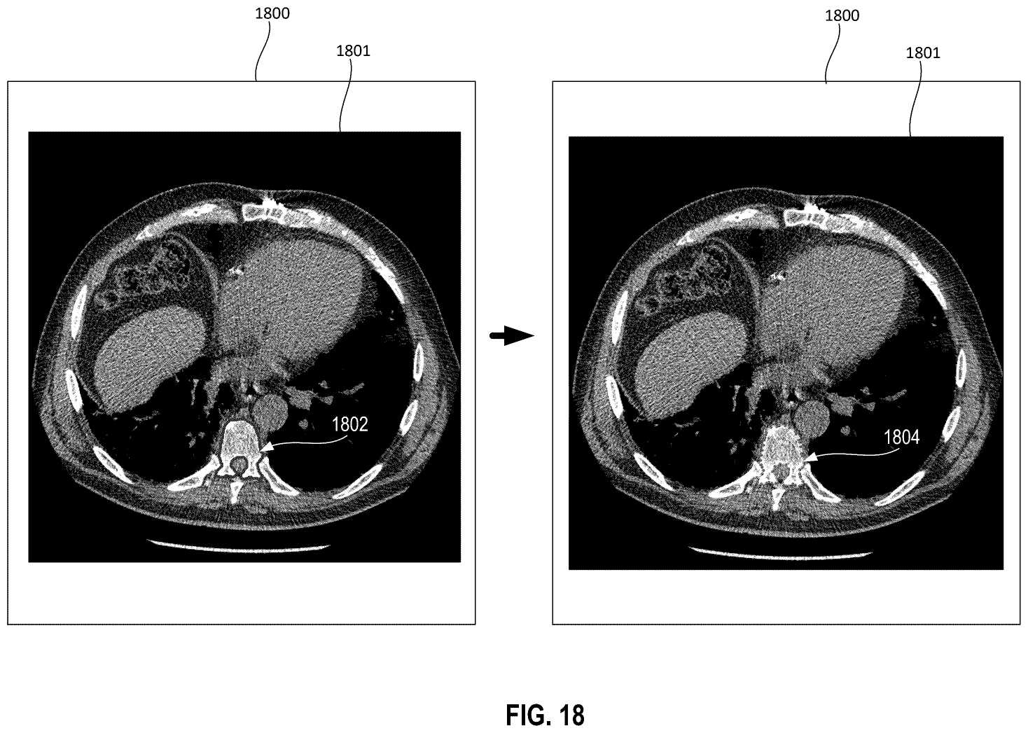

FIG. 18 is an illustration of a graphical user interface transforming the display of a boundary to anchor indicators in accordance with one or more example embodiments. On the left, graphical user interface 1800 displays image 1801. Image 1801 is a high-resolution image (e.g., a CT scan of a subject's chest. Image 1801 is displayed with boundary 1802 that identifies a region of bone tissue (e.g., spine).

The image processor 108 described in FIGS. 1A-1B may be used to transform the display of boundary 1802 to a display of anchor indicators 1804 over image 1801 as shown on the right. The anchor indicators 1804, when connected via line segments as shown, approximate boundary 1802 within selected tolerances. Anchor indicators 1804, which may be one example of an implementation for anchor indicators 144 in FIG. 1B, represent anchor points. Each of anchor indicators 1804 is a movable graphical indicator that can be moved via user input to adjust the position of a corresponding anchor point.

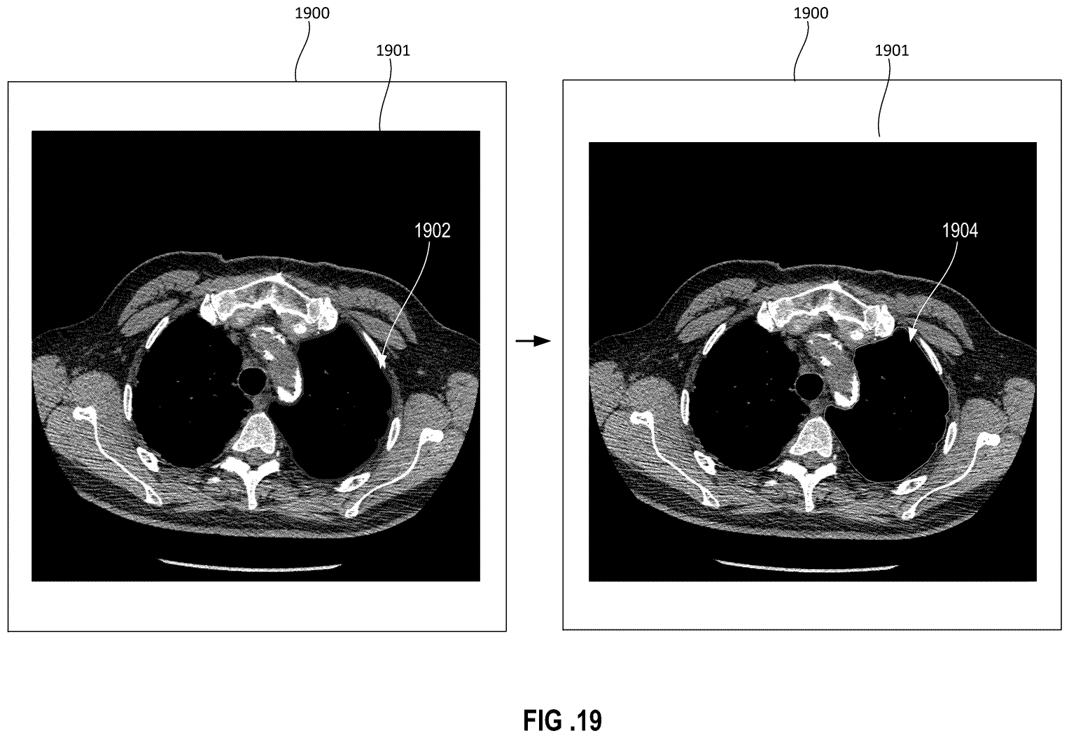

FIG. 19 is an illustration of a graphical user interface transforming the display of a boundary to anchor indicators in accordance with one or more example embodiments. On the left, graphical user interface 1900 displays image 1901. Image 1901 is a high-resolution image (e.g., a CT scan of a subject's chest.) Image 1901 is displayed with boundary 1902 that identifies a region of lung tissue (e.g., lung lobe(s)).

The image processor 108 described in FIGS. 1A-1B may be used to transform the display of boundary 1902 to a display of anchor indicators 1904 over image 1901 as shown on the right. The anchor indicators 1904, when connected via line segments as shown, approximate boundary 1902 within selected tolerances. Anchor indicators 1904, which may be one example of an implementation for anchor indicators 144 in FIG. 1B, represent anchor points. Each of anchor indicators 1904 is a movable graphical indicator that can be moved via user input to adjust the position of a corresponding anchor point.

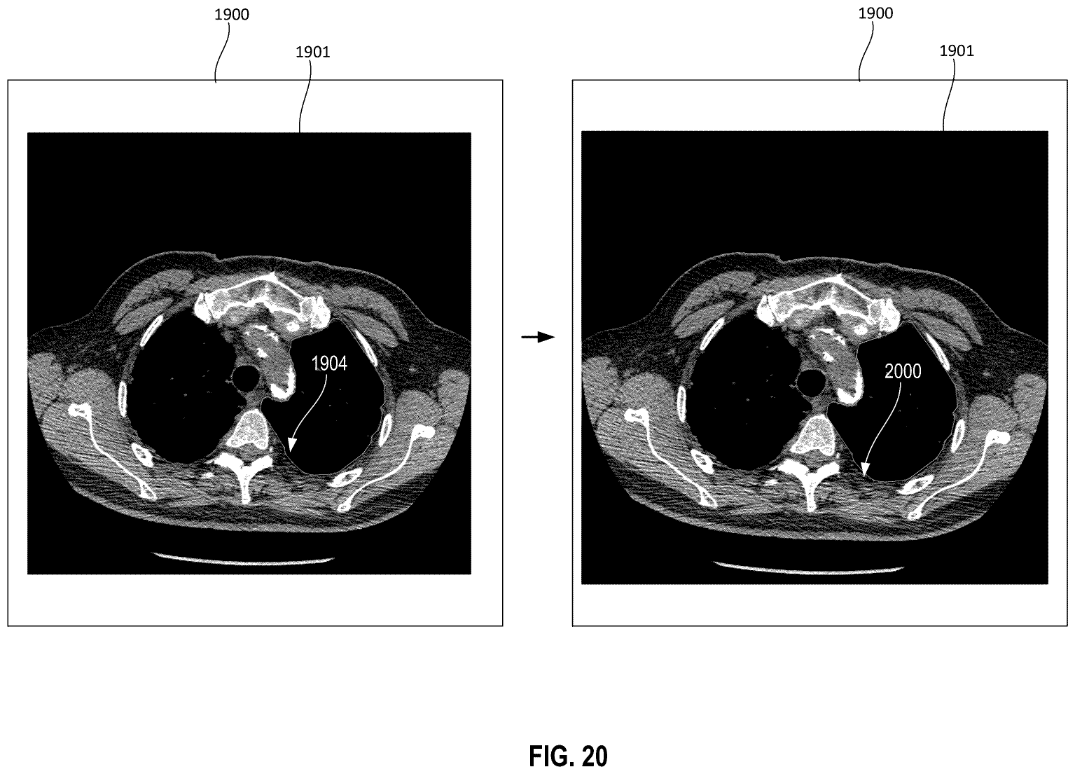

FIG. 20 is an illustration of the graphical user interface from FIG. 19 showing a change in anchor point density in accordance with one or more example embodiments. The anchor point density is changed in response to a change in the selected threshold used to evaluate perpendicular distances as described with respect to the embodiments herein (e.g., as discussed in FIGS. 2A, 2B, 3A, and 3B). On the left, anchor indicators 1904 from FIG. 19 are shown. Lowering the selected threshold used to compute the perpendicular distances described herein reduces the density of anchor points that are selected and thereby, the number of anchor indicators displayed, as shown on the right with anchor indicators 2000.

### V. Example Computing System

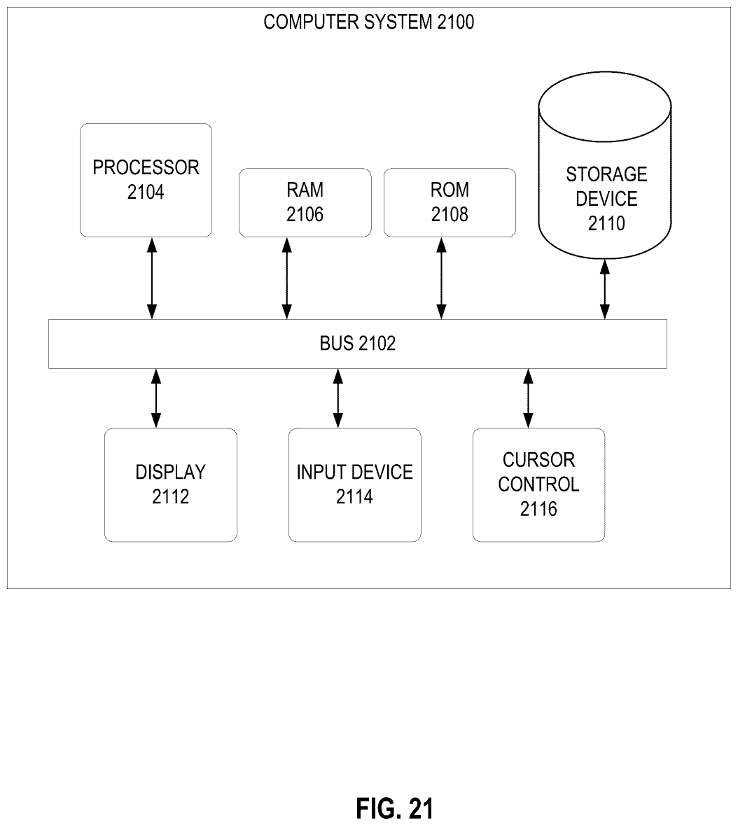

FIG. 21 is a block diagram illustrating an example of a computing system, in accordance with one or more example embodiments. Computing system 2100 may be used to implement computing platform 102 and/or remote system 107 in FIG. 1A and/or any components therein.

In one or more examples, computer system 2100 can include a bus 2102 or other communication mechanism for communicating information, and a processor 2104 coupled with bus 2102 for processing information. In various embodiments, computer system 2100 can also include a memory, which can be a random-access memory (RAM) 2106 or other dynamic storage device, coupled to bus 2102 for determining instructions to be executed by processor 2104. Memory also can be used for storing temporary variables or other intermediate information during execution of instructions to be executed by processor 2104. In various embodiments, computer system 2100 can further include a read only memory (ROM) 2108 or other static storage device coupled to bus 2102 for storing static information and instructions for processor 2104. A storage device 2110, such as a magnetic disk or optical disk, can be provided and coupled to bus 2102 for storing information and instructions.

In various embodiments, computer system 2100 can be coupled via bus 2102 to a display 2112, such as a cathode ray tube (CRT) or liquid crystal display (LCD), for displaying information to a computer user. An input device 2114, including alphanumeric and other keys, can be coupled to bus 2102 for communicating information and command selections to processor 2104. Another type of user input device is a cursor control 2116, such as a mouse, a joystick, a trackball, a gesture input device, a gaze-based input device, or cursor direction keys for communicating direction information and command selections to processor 2104 and for controlling cursor movement on display 2112. This input device 2114 typically has two degrees of freedom in two axes, a first axis (e.g., x) and a second axis (e.g., y), that allows the device to specify positions in a plane. However, it should be understood that input devices 2114 allowing for three-dimensional (e.g., x, y, and z) cursor movement are also contemplated herein.

Consistent with certain implementations of the present teachings, results can be provided by computer system 2100 in response to processor 2104 executing one or more sequences of one or more instructions contained in RAM 2106. Such instructions can be read into RAM 2106 from another computer-readable medium or computer-readable storage medium, such as storage device 2110. Execution of the sequences of instructions contained in RAM 2106 can cause processor 2104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 2104 for execution. Such a medium can take many forms, including but not limited to, non-volatile media, volatile media, and transmission media. Examples of non-volatile media can include, but are not limited to, optical, solid state, magnetic disks, such as storage device 2110. Examples of volatile media can include, but are not limited to, dynamic memory, such as RAM 2106. Examples of transmission media can include, but are not limited to, coaxial cables, copper wire, and fiber optics, including the wires that comprise bus 2102.

Common forms of computer-readable media include, for example, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 2104 of computer system 2100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 2100, whereby processor 2104 would execute the analyses and determinations provided by these engines, subject to instructions provided by any one of, or a combination of, the memory components RAM 2106, ROM, 2108, or storage device 2110 and user input provided via input device 2114.

In some example embodiments, the computing system 2100 can be used to execute various interactive computer software applications that can be used for organization, analysis and/or storage of data in various formats. Alternatively, the computing system 2100 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 2114. The user interface can be generated and presented to a user by the computing system 2100 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

### VI. Recitation of Exemplary Embodiments

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Embodiment 1. A method comprising: receiving an image associated with a portion of anatomy of a subject; extracting a plurality of boundary points for an initial boundary associated with a corresponding region of pixels in the image, wherein the plurality of boundary points is associated with a sequential order for a selected two-dimensional plane corresponding to the image; evaluating the plurality of boundary points according to the sequential order to select a plurality of anchor points from the plurality of boundary points; and generating an anchor point image for display in a graphical user interface on a display device, wherein the anchor point image includes a plurality of anchor indicators that represent the plurality of anchor points. The evaluating comprises: determining that a first boundary point in the plurality of boundary points is a first anchor point; and determining that a current boundary point of the plurality of boundary points being evaluated is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold; and

Embodiment 2. The method of embodiment 1, wherein the evaluating further comprises: determining that the current boundary point being evaluated is not a next anchor point in the plurality of anchor points when no perpendicular distance computed for the portion of the initial boundary located between the previous anchor point and the current boundary point with respect to the line extending between the previous anchor point and the current boundary point is greater than the selected threshold.

Embodiment 3. The method of embodiment 1 or embodiment 2, wherein the evaluating further comprises: determining that the current boundary point being evaluated is not a next anchor point in the plurality of anchor points when no boundary points according to the sequential order are present between the previous anchor point and the current boundary point being evaluated.

Embodiment 4. The method of any one of embodiments 1-3, further comprising: receiving user input that moves at least one anchor indicator of the plurality of anchor indicators in the graphical user interface.

Embodiment 5. The method of embodiment 4, further comprising: adjusting an original position of at least one anchor point in the plurality of anchor points that corresponds to the at least one anchor indicator based on the user input such that the plurality of anchor points have final positions.

Embodiment 6. The method of embodiment 4 or embodiment 5, further comprising: generating adjustment data based on the user input, wherein the adjustment data includes an adjusted segmented image in which at least some pixels of the segmented image are reclassified to form the adjusted segmented image based on the final positions of the plurality of anchor points.

Embodiment 7. The method of embodiment 6, further comprising: retraining a segmentation model that comprises a machine learning model using the adjusted segmented image.

Embodiment 8. The method of any one of embodiments 1-7, wherein the evaluating further comprises: determining that a last boundary point in the plurality of boundary points is a last anchor point.

Embodiment 9. The method of any one of embodiments 1-8, wherein determining that the current boundary point of the plurality of boundary points being evaluated is the next anchor point in the plurality of anchor points comprises: computing a perpendicular distance between each middle boundary point of the plurality of boundary points located between the previous anchor point and the current boundary point and a line extending between the previous anchor point and the current boundary point to form a set of perpendicular distances; determining that at least one perpendicular distance of the set of perpendicular distances is greater than the selected threshold; and determining that the current boundary point is to be the next anchor point in response to a determination that the at least one perpendicular distance of the set of perpendicular distances is greater than the selected threshold.

Embodiment 10. The method of any one of embodiments 1-9, wherein the selected threshold is selected to be between 1 and 10 pixel units.

Embodiment 11. The method of any one of embodiments 1-10, wherein the anchor point image displayed in the graphical user interface further includes a new boundary that is formed by line segments connecting the plurality of anchor indicators that represent the plurality of anchor points.

Embodiment 12. The method of any one of embodiments 1-11, further comprising: receiving user input that moves at least one anchor indicator of the plurality of anchor indicators in the graphical user interface; adjusting the initial boundary to form a new boundary based on the user input; and generating an adjusted image in which the new boundary is overlaid over the image; and forming an input for an algorithm using the adjusted image.

Embodiment 13. The method of any one of embodiments 1-12, wherein the image comprises a magnetic resonance imaging (MRI) image, a computed tomography (CT) image, an X-ray image, an ultrasound image, a positron-emission tomography (PET) image, a single-photon emission computerized tomography (SPECT) image, or an optical coherence tomography (OCT) image.

Embodiment 14. The method of any one of embodiments 1-13, wherein the image is a two-dimensional image or a three-dimensional image.

Embodiment 15. A method comprising: receiving a plurality of images associated with a portion of anatomy of a subject; performing segmentation using the plurality of images and a segmentation model to generate a plurality of segmented images; extracting, for each segmented image of the plurality of segmented images, a plurality of boundary points for each initial boundary of a set of initial boundary associated with each segmented image, wherein the plurality of boundary points is associated with a sequential order for a selected two-dimensional plane corresponding to the plurality of images; evaluating, for each segmented image of the plurality of segmented images, the plurality of boundary points for each initial boundary of the set of initial boundary associated with each segmented image according to the sequential order to select a plurality of anchor points from the plurality of boundary points for each initial boundary of the set of initial boundary identified in each image; and generating an anchor point image for display in a graphical user interface on a display device, wherein the anchor point image includes a plurality of anchor indicators that represent the plurality of anchor points and a plurality of line segments connecting the plurality of anchor indicators. The evaluating comprises: determining that a first boundary point in the plurality of boundary points is a first anchor point; determining that a last boundary point in the plurality of boundary points is a last anchor point; and determining that a current boundary point of the plurality of boundary points being evaluated is a next anchor point in the plurality of anchor points when at least one perpendicular distance of a set of perpendicular distances computed for a corresponding set of middle boundary points with respect to a line extending between a previous anchor point and the current boundary point is greater than a selected threshold;

Embodiment 16. The method of embodiment 15, wherein the segmentation model comprises a machine learning model and further comprising: receiving user input that adjust a position of at least one of the plurality of anchor indicators; adjusting an original position of at least one anchor point in the plurality of anchor points corresponding to the at least one of the plurality of anchor indicators to form final positions for the plurality of anchor points; and modifying a corresponding segmented image of the plurality of segmented images using the final positions of the plurality of anchor points to form an adjusted segmented image for use in retraining the segmentation model.

Embodiment 17. A system including one or more computing devices, comprising: one or more non-transitory computer-readable storage media including instructions; and one or more processors coupled to the one or more storage media. The one or more processors configured to execute the instructions to: receive an image associated with a portion of anatomy of a subject; extract a plurality of boundary points for an initial boundary associated with a corresponding region of pixels in the image, wherein the plurality of boundary points is associated with a sequential order for a selected two-dimensional plane corresponding to the image; evaluate the plurality of boundary points according to the sequential order to select a plurality of anchor points from the plurality of boundary points; and generate an anchor point image for display in a graphical user interface on a display device, wherein the anchor point image includes a plurality of anchor indicators that represent the plurality of anchor points. The evaluating comprises: determining that a first boundary point in the plurality of boundary points is a first anchor point; and determining that a current boundary point of the plurality of boundary points being evaluated is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold.

Embodiment 18. The system of embodiment 17, wherein the one or more processors is configured to execute the instructions determine that the current boundary point being evaluated is not a next anchor point in the plurality of anchor points when no boundary points are present along the initial boundary between the previous anchor point and the current boundary point being evaluated.

Embodiment 19. The system of embodiment 17 or embodiment 18, wherein the one or more processors is further configured to execute the instructions to: receive user input that moves at least one anchor indicator of the plurality of anchor indicators in the graphical user interface.

Embodiment 20. The system of embodiment 19, wherein the one or more processors is further configured to execute the instructions to: adjust an original position of at least one anchor point in the plurality of anchor points that corresponds to the at least one anchor indicator based on the user input to form final positions for the plurality of anchor points.

Embodiment 21. The system of embodiment 19 or embodiment 20, wherein the one or more processors is further configured to execute the instructions to: generate adjustment data based on the user input, wherein the adjustment data includes an adjusted segmented image in which at least some pixels of the segmented image are reclassified to form the adjusted segmented image based on the final positions of the plurality of anchor points.

Embodiment 22. The system of embodiment 21, wherein the one or more processors is further configured to execute the instructions to: retrain a segmentation model that comprises a machine learning model using the adjusted segmented image.

Embodiment 23. The system of any one of embodiments 16-22, wherein the one or more processors is further configured to execute the instructions to: determine that a last boundary point in the plurality of boundary points is a last anchor point.

Embodiment 24. The system of any one of embodiments 16-23, wherein the one or more processors is further configured to execute the instructions to: compute a perpendicular distance between each middle boundary point of the plurality of boundary points located between the previous anchor point and the current boundary point and a line extending between the previous anchor point and the current boundary point to form a set of perpendicular distances; determine that at least one perpendicular distance of the set of perpendicular distances is greater than the selected threshold; and determine that the current boundary point is to be the next anchor point in response to a determination that the at least one perpendicular distance of the set of perpendicular distances is greater than the selected threshold.

Embodiment 25. The system of any one of embodiments 16-24, wherein the selected threshold is selected to be between 1 and 10 pixel units.

Embodiment 26. The system of any one of embodiments 16-25, wherein the anchor point image displayed in the graphical user interface further includes a new boundary that is formed by line segments connecting the plurality of anchor indicators that represent the plurality of anchor points.

Embodiment 27. The system of any one of embodiments 16-26, wherein the image comprises a magnetic resonance imaging (MRI) image, a computed tomography (CT) image, an X-ray image, an ultrasound image, a positron-emission tomography (PET) image, a single-photon emission computerized tomography (SPECT) image, or an optical coherence tomography (OCT) image.

Embodiment 28. The system of any one of embodiments 16-27, wherein the image is a two-dimensional image or a three-dimensional image.

### Additional Embodiments

Embodiment A1: A method, by one or more computing devices: receiving a medical image of a patient; segmenting the medical image into plurality of regions of pixels, wherein the plurality of regions of pixels comprises at least a first region of pixels and a second region of pixels; extracting a set of boundary points along an identified boundary between the first region of pixels and the second region of pixels; and determining sequential pairs of anchor points, wherein the sequential pairs of anchor points are configured to be utilized to adjust the identified boundary between the first region of pixels and the second region of pixels, and wherein determining the sequential pairs of anchor points comprises: identifying a starting anchor point, wherein: for a first one of an identified pairs of boundary points, the starting anchor point is the first boundary point in the set of boundary points, and for subsequent ones of the identified pairs of boundary points, the starting anchor point is the ending anchor point of an immediately preceding identified pair of boundary points; and identifying an ending anchor point, wherein a curvature between the starting anchor point and the ending anchor point satisfies a predetermined criterion.

Embodiment A2: The method of Embodiment A1, further comprising: causing a display of one or more other computing devices to display a second image based on the sequential pairs of anchor points; and in response to determining one or more user inputs corresponding to a manipulation of at least one anchor point of the sequential pairs of anchor points, adjusting the identified boundary between the first region of pixels and the second region of pixels.

Embodiment A3: The method of Embodiment A1, wherein to adjust the identified boundary between the first region of pixels and the second region of pixels is to adjust a contour of the identified boundary.

Embodiment A4: The method of Embodiment A1, wherein to adjust the identified boundary between the first region of pixels and the second region of pixels is to correct a class label corresponding to the first region of pixels or the second region of pixels.

Embodiment A5: The method of Embodiment A4, wherein to correct the class label corresponding to the first region of pixels or the second region of pixels is performed in response to receiving one or more user inputs from a human pathologist.

Embodiment A6: The method of Embodiment A1, wherein the curvature between the starting anchor point and the ending anchor point comprises a perpendicular distance between the starting anchor point and the ending anchor point.

Embodiment A7: The method of Embodiment A1, wherein the curvature between the starting anchor point and the ending anchor point satisfies the predetermined criterion when the curvature exceeds a perpendicular distance threshold.

Embodiment A8: The method of Embodiment A7, wherein the perpendicular distance threshold comprises a perpendicular distance of approximately one pixel.

Embodiment A9: The method of Embodiment A7, wherein the perpendicular distance threshold comprises a perpendicular distance of approximately two pixels.

Embodiment A10: The method of Embodiment A7, wherein the perpendicular distance threshold comprises a user-configurable threshold.

Embodiment A11: The method of Embodiment A10, wherein the user-configurable threshold is configured to be adjusted so as to adjust the identified boundary between the first region of pixels and the second region of pixels in accordance with a desired accuracy.

Embodiment A12: The method of Embodiment A11, wherein the desired accuracy varies based on a total number of the sequential pairs of anchor points.

Embodiment A13: The method of Embodiment A1, wherein, for a last one of the identified pairs of boundary points, the ending anchor point is the last boundary point in the set of boundary points.

Embodiment A14: The method of Embodiment A1, wherein determining the sequential pairs of anchor points further comprises: forgoing identifying the ending anchor point when the curvature between the starting anchor point and the ending anchor point fails to satisfy the predetermined criterion.

Embodiment A15: The method of Embodiment A1, wherein identifying the plurality of regions of pixels in the image comprises segmenting regions of pixels indicative of a normal region and regions of pixels indicative of a disease region.

Embodiment A16: The method of Embodiment A1, wherein identifying the plurality of regions of pixels in the image further comprises: inputting the image to a machine-learning model trained to segment the plurality of regions of pixels in the image; and utilizing the machine-learning model to: segment at least the first region of pixels and the second region of pixels; and output a predicted class label for each of the first region of pixels and the second region of pixels.

Embodiment A17: The method of Embodiment A16, further comprising: displaying a second image based on the predicted class label for each of the first region of pixels and the second region of pixels; receiving one or more user inputs corresponding to a request to update the predicted class label for at least one of the first region of pixels or the second region of pixels; and displaying a third image based on the updated predicted class label.

Embodiment A18: The method of Embodiment A17, further comprising inputting the third image to the machine-learning model for retraining the machine-learning model.

Embodiment A19: The method of Embodiment A1, further comprising determining another one of the sequential pairs of anchor points, wherein determining the other one of the sequential pairs of anchor points comprises: identifying another starting anchor point, wherein the other starting anchor point is the ending anchor point of the first one of the identified pairs of boundary points; and identifying another ending anchor point when a curvature between the other starting anchor point and the other ending anchor point satisfies the predetermined criterion.

Embodiment A20: The method of Embodiment A19, wherein determining the other one of the sequential pairs of anchor points further comprises: forgoing identifying the other ending anchor point when the curvature between the other starting anchor point and the other ending anchor point fails to satisfy the predetermined criterion.

Embodiment A21: The method of Embodiment A1, wherein the image comprises an image of one or more lesions, and wherein the sequential pairs of anchor points are configured to be utilized to adjust the identified boundary to accurately label the one or more lesions.

Embodiment A22: The method of Embodiment A1, wherein to adjust the identified boundary utilizing the sequential pairs of anchor points is to render the medical image suitable for use in a clinical trial of one or more pharmaceutical products.

Embodiment A23: The method of Embodiment A1, wherein the image comprises a magnetic resonance imaging (MRI) image, a computed tomography (CT) image, an X-ray image, an ultrasound image, a positron-emission tomography (PET) image, a single-photon emission computerized tomography (SPECT) image, or an optical coherence tomography (OCT) image.

Embodiment A24: A system including one or more computing devices, comprising: one or more non-transitory computer-readable storage media including instructions; and one or more processors coupled to the one or more storage media, the one or more processors configured to execute the instructions to: receive an image, wherein the image comprises a medical image of a patient; identify a plurality of regions of pixels in the image, wherein the plurality of regions of pixels comprises at least a first region of pixels and a second region of pixels; extract a set of boundary points along an identified boundary between the first region of pixels and the second region of pixels; and determine sequential pairs of anchor points, wherein the sequential pairs of anchor points are configured to be utilized to adjust the identified boundary between the first region of pixels and the second region of pixels, and wherein the instructions to determine one of the sequential pairs of anchor points further comprises instructions to: identify a starting anchor point, wherein: for a first one of identified pairs of boundary points, the starting anchor point is the first boundary point in the set of boundary points, and for subsequent ones of the identified pairs of boundary points, the starting anchor point is the ending anchor point of an immediately preceding identified pair of boundary points; and identify an ending anchor point, wherein a curvature between the starting anchor point and the ending anchor point satisfies a predetermined criterion.

Embodiment A25: The system of Embodiment A24, wherein the instructions further comprises instructions to: cause a display of one or more other computing devices to display a second image based on the sequential pairs of anchor points; and in response to determining one or more user inputs corresponding to a manipulation of at least one anchor point of the sequential pairs of anchor points, adjust the identified boundary between the first region of pixels and the second region of pixels.

Embodiment A26: The system of Embodiment A24, wherein to adjust the identified boundary between the first region of pixels and the second region of pixels is to adjust a contour of the identified boundary.

Embodiment A27: The system of Embodiment A24, wherein to adjust the identified boundary between the first region of pixels and the second region of pixels is to correct a class label corresponding to the first region of pixels or the second region of pixels.

Embodiment A28: The system of Embodiment A27, wherein to correct the class label corresponding to the first region of pixels or the second region of pixels is performed in response to receiving one or more user inputs from a human pathologist.

Embodiment A29: The system of Embodiment A24, wherein the curvature between the starting anchor point and the ending anchor point comprises a perpendicular distance between the starting anchor point and the ending anchor point.

Embodiment A30: The system of Embodiment A24, wherein the curvature between the starting anchor point and the ending anchor point satisfies the predetermined criterion when the curvature exceeds a perpendicular distance threshold.

Embodiment A31: The system of Embodiment A30, wherein the perpendicular distance threshold comprises a perpendicular distance of approximately one pixel.

Embodiment A32: The system of Embodiment A30, wherein the perpendicular distance threshold comprises a perpendicular distance of approximately two pixels.

Embodiment A33: The system of Embodiment A30, wherein the perpendicular distance threshold comprises a user-configurable threshold.

Embodiment A34: The system of Embodiment A33, wherein the user-configurable threshold is configured to be adjusted so as to adjust the identified boundary between the first region of pixels and the second region of pixels in accordance with a desired accuracy.

Embodiment A35: The system of Embodiment A34, wherein the desired accuracy varies based on a total number of the sequential pairs of anchor points.

Embodiment A36: The system of Embodiment A24, wherein, for a last one of the identified pairs of boundary points, the ending anchor point is the last boundary point in the set of boundary points.

Embodiment A37: The system of Embodiment A24, wherein the instructions to determine the one of the sequential pairs of anchor points further comprises instructions to: forgo identifying the ending anchor point when the curvature between the starting anchor point and the ending anchor point fails to satisfy the predetermined criterion.

Embodiment A38: The system of Embodiment A24, wherein the instructions to identify the plurality of regions of pixels in the image further comprises instructions to segment regions of pixels indicative of a normal region and regions of pixels indicative of a disease region.

Embodiment A39: The system of Embodiment A24, wherein the instructions to identify the plurality of regions of pixels in the image further comprises instructions to: input the image to a machine-learning model trained to segment the plurality of regions of pixels in the image; and utilize the machine-learning model to: segment at least the first region of pixels and the second region of pixels; and output a predicted class label for each of the first region of pixels and the second region of pixels.

Embodiment A40: The system of Embodiment A39, wherein the instructions further comprises instructions to: display a second image based on the predicted class label for each of the first region of pixels and the second region of pixels; receive one or more user inputs corresponding to a request to update the predicted class label for at least one of the first region of pixels or the second region of pixels; and display a third image based on the updated predicted class label.

Embodiment A41: The system of Embodiment A40, wherein the instructions further comprises instructions to input the third image to the machine-learning model for retraining the machine-learning model.

Embodiment A42: The system of Embodiment A24, wherein the instructions further comprises instructions to determine another one of the sequential pairs of anchor points, wherein the instructions to determine the other one of the sequential pairs of anchor points further comprises instructions to: identify another starting anchor point, wherein the other starting anchor point is the ending anchor point of the first one of the identified pairs of boundary points; and identify another ending anchor point when a curvature between the other starting anchor point and the other ending anchor point satisfies the predetermined criterion.

Embodiment A43: The system of Embodiment A42, wherein the instructions to determine the other one of the sequential pairs of anchor points further comprises instructions to: forgo identifying the other ending anchor point when the curvature between the other starting anchor point and the other ending anchor point fails to satisfy the predetermined criterion.

Embodiment A44: The system of Embodiment A24, wherein the image comprises an image of one or more lesions, and wherein the sequential pairs of anchor points are configured to be utilized to adjust the identified boundary to accurately label the one or more lesions.

Embodiment A45: The system of Embodiment A24, wherein to adjust the identified boundary utilizing the sequential pairs of anchor points is to render the medical image suitable for use in a clinical trial of one or more pharmaceutical products.

Embodiment A46: The system of Embodiment A24, wherein the image comprises a magnetic resonance imaging (MRI) image, a computed tomography (CT) image, an X-ray image, an ultrasound image, a positron-emission tomography (PET) image, a single-photon emission computerized tomography (SPECT) image, or an optical coherence tomography (OCT) image.

Embodiment A47: A non-transitory computer-readable medium comprising instructions that, when executed by one or more processors of one or more computing devices, cause the one or more processors to: receive an image, wherein the image comprises a medical image of a patient; identify a plurality of regions of pixels in the image, wherein the plurality of regions of pixels comprises at least a first region of pixels and a second region of pixels; extract a set of boundary points along an identified boundary between the first region of pixels and the second region of pixels; and determine sequential pairs of anchor points, wherein the sequential pairs of anchor points are configured to be utilized to adjust the identified boundary between the first region of pixels and the second region of pixels, and wherein the instructions to determine one of the sequential pairs of anchor points further comprises instructions to: identify a starting anchor point, wherein: for a first one of the identified pairs of boundary points, the starting anchor point is the first boundary point in the set of boundary points, and for subsequent ones of the identified pairs of boundary points, the starting anchor point is the ending anchor point of an immediately preceding identified pair of boundary points; and identify an ending anchor point, wherein a curvature between the starting anchor point and the ending anchor point satisfies a predetermined criterion.

Embodiment A48: The non-transitory computer-readable medium of Embodiment A47, wherein the instructions further comprises instructions to: cause a display of one or more other computing devices to display a second image based on the sequential pairs of anchor points; and in response to determining one or more user inputs corresponding to a manipulation of at least one anchor point of the sequential pairs of anchor points, adjust the identified boundary between the first region of pixels and the second region of pixels.

Embodiment A49: The non-transitory computer-readable medium of Embodiment A47, wherein to adjust the identified boundary between the first region of pixels and the second region of pixels is to adjust a contour of the identified boundary.

Embodiment A50: The non-transitory computer-readable medium of Embodiment A47, wherein to adjust the identified boundary between the first region of pixels and the second region of pixels is to correct a class label corresponding to the first region of pixels or the second region of pixels.

Embodiment A51: The non-transitory computer-readable medium of Embodiment A50, wherein to correct the class label corresponding to the first region of pixels or the second region of pixels is performed in response to receiving one or more user inputs from a human pathologist.

Embodiment A52: The non-transitory computer-readable medium of Embodiment A47, wherein the curvature between the starting anchor point and the ending anchor point comprises a perpendicular distance between the starting anchor point and the ending anchor point.

Embodiment A53: The non-transitory computer-readable medium of Embodiment A47, wherein the curvature between the starting anchor point and the ending anchor point satisfies the predetermined criterion when the curvature exceeds a perpendicular distance threshold.

Embodiment A54: The non-transitory computer-readable medium of Embodiment A53, wherein the perpendicular distance threshold comprises a perpendicular distance of approximately one pixel.

Embodiment A55: The non-transitory computer-readable medium of Embodiment A53, wherein the perpendicular distance threshold comprises a perpendicular distance of approximately two pixels.

Embodiment A56: The non-transitory computer-readable medium of Embodiment A53, wherein the perpendicular distance threshold comprises a user-configurable threshold.

Embodiment A57: The non-transitory computer-readable medium of Embodiment A56, wherein the user-configurable threshold is configured to be adjusted so as to adjust the identified boundary between the first region of pixels and the second region of pixels in accordance with a desired accuracy.

Embodiment A58: The non-transitory computer-readable medium of Embodiment A57, wherein the desired accuracy varies based on a total number of the sequential pairs of anchor points.

Embodiment A59: The non-transitory computer-readable medium of Embodiment A47, wherein, for a last one of the identified pairs of boundary points, the ending anchor point is the last boundary point in the set of boundary points.

Embodiment A60: The non-transitory computer-readable medium of Embodiment A47, wherein the instructions to determine the one of the sequential pairs of anchor points further comprises instructions to: forgo identifying the ending anchor point when the curvature between the starting anchor point and the ending anchor point fails to satisfy the predetermined criterion.

Embodiment A61: The non-transitory computer-readable medium of Embodiment A47, wherein the instructions to identify the plurality of regions of pixels in the image further comprises instructions to segment regions of pixels indicative of a normal region and regions of pixels indicative of a disease region.

Embodiment A62: The non-transitory computer-readable medium of Embodiment A47, wherein the instructions to identify the plurality of regions of pixels in the image further comprises instructions to: input the image to a machine-learning model trained to segment the plurality of regions of pixels in the image; and utilize the machine-learning model to: segment at least the first region of pixels and the second region of pixels; and output a predicted class label for each of the first region of pixels and the second region of pixels.

Embodiment A63: The non-transitory computer-readable medium of Embodiment A62, wherein the instructions further comprises instructions to: display a second image based on the predicted class label for each of the first region of pixels and the second region of pixels; receive one or more user inputs corresponding to a request to update the predicted class label for at least one of the first region of pixels or the second region of pixels; and display a third image based on the updated predicted class label.

Embodiment A64: The non-transitory computer-readable medium of Embodiment A63, wherein the instructions further comprises instructions to input the third image to the machine-learning model for retraining the machine-learning model.

Embodiment A65: The non-transitory computer-readable medium of Embodiment A47, wherein the instructions further comprises instructions to determine another one of the sequential pairs of anchor points, wherein the instructions to determine the other one of the sequential pairs of anchor points further comprises instructions to: identify another starting anchor point, wherein the other starting anchor point is the ending anchor point of the first one of the identified pairs of boundary points; and identify another ending anchor point when a curvature between the other starting anchor point and the other ending anchor point satisfies the predetermined criterion.

Embodiment A66: The non-transitory computer-readable medium of Embodiment A65, wherein the instructions to determine the other one of the sequential pairs of anchor points further comprises instructions to: forgo identifying the other ending anchor point when the curvature between the other starting anchor point and the other ending anchor point fails to satisfy the predetermined criterion.

Embodiment A67: The non-transitory computer-readable medium of Embodiment A47, wherein the image comprises an image of one or more lesions, and wherein the sequential pairs of anchor points are configured to be utilized to adjust the identified boundary to accurately label the one or more lesions.

Embodiment A68: The non-transitory computer-readable medium of Embodiment A47, wherein to adjust the identified boundary utilizing the sequential pairs of anchor points is to render the medical image suitable for use in a clinical trial of one or more pharmaceutical products.

Embodiment A69: The non-transitory computer-readable medium of Embodiment A47, wherein the image comprises a magnetic resonance imaging (MRI) image, a computed tomography (CT) image, an X-ray image, an ultrasound image, a positron-emission tomography (PET) image, a single-photon emission computerized tomography (SPECT) image, or an optical coherence tomography (OCT) image.

### VII. Example Definitions and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for case of review only and do not limit any combination of elements discussed.

The term “subject” may refer to a subject of a clinical trial, a person or animal undergoing treatment, a person or animal undergoing anti-cancer therapies, a person or animal being monitored for remission or recovery, a person or animal undergoing a preventative health analysis (e.g., due to their medical history), or any other person or patient or animal of interest. In various cases, “subject” and “patient” may be used interchangeably herein.

The term “OCT image” may refer to an image of a tissue, an organ, etc., such as a retina, that is scanned or captured using optical coherence tomography (OCT) imaging technology. The term may refer to one or both of 2D “slice” images and 3D “volume” images. When not explicitly indicated, the term may be understood to include OCT volume images.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items. As used herein, the term “subset” includes one or more of the items included in the referenced set. For example, a subset may include one item in the referenced set or may include all items in the referenced set.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B;

item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning (ML) algorithms, or a combination thereof.

As used herein, “machine learning” may include the practice of using algorithms to parse data, learn from it, and then make a determination or prediction about something in the world. Machine learning uses algorithms that can learn from data without relying on rules-based programming.

As used herein, an “artificial neural network” or “neural network” may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in, for example, two ways; when it is being trained (e.g., using a training dataset) it is in training mode and when it puts what it has learned into practice (e.g., using a test dataset) it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs.

A neural network may process information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### VIII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Further, the subject matter described herein can be embodied in systems, apparatus, methods, and/or articles depending on the desired configuration. The implementations set forth in the description herein do not represent all implementations consistent with the subject matter described. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method comprising:
receiving an image associated with a portion of anatomy of a subject;
extracting a plurality of boundary points for an initial boundary associated with a corresponding region of pixels in the image, wherein the plurality of boundary points is associated with a sequential order for a selected two-dimensional plane corresponding to the image;
evaluating the plurality of boundary points according to the sequential order to select a plurality of anchor points from the plurality of boundary points, wherein the evaluating comprises:
determining that a first boundary point in the plurality of boundary points is a first anchor point; and
determining that a current boundary point of the plurality of boundary points being evaluated is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold; and
generating an anchor point image for display in a graphical user interface on a display device, wherein the anchor point image includes a plurality of anchor indicators that represent the plurality of anchor points.

2. The method of claim 1, wherein the evaluating further comprises:
determining that the current boundary point being evaluated is not a next anchor point in the plurality of anchor points when no perpendicular distance computed for the portion of the initial boundary located between the previous anchor point and the current boundary point with respect to the line extending between the previous anchor point and the current boundary point is greater than the selected threshold and/or no boundary points according to the sequential order are present between the previous anchor point and the current boundary point being evaluated.

3. The method of claim 1, further comprising:
receiving user input that moves at least one anchor indicator of the plurality of anchor indicators in the graphical user interface;
adjusting an original position of at least one anchor point in the plurality of anchor points that corresponds to the at least one anchor indicator based on the user input such that the plurality of anchor points have final positions; and
generating adjustment data based on the user input, wherein the adjustment data includes an adjusted segmented image in which at least some pixels of a segmented image are reclassified to form the adjusted segmented image based on the final positions of the plurality of anchor points.

4. The method of claim 3, further comprising:
retraining a segmentation model that comprises a machine learning model using the adjusted segmented image.

5. The method of claim 1, wherein the evaluating further comprises:
determining that a last boundary point in the plurality of boundary points is a last anchor point;
wherein determining that the current boundary point of the plurality of boundary points being evaluated is the next anchor point in the plurality of anchor points comprises:
computing a perpendicular distance between each middle boundary point of the plurality of boundary points located between the previous anchor point and the current boundary point and a line extending between the previous anchor point and the current boundary point to form a set of perpendicular distances;
determining that at least one perpendicular distance of the set of perpendicular distances is greater than the selected threshold; and
determining that the current boundary point is to be the next anchor point in response to a determination that the at least one perpendicular distance of the set of perpendicular distances is greater than the selected threshold.

6. The method of claim 1, wherein the selected threshold is selected to be between 1 and 10 pixel units.

7. The method of claim 1, wherein the anchor point image displayed in the graphical user interface further includes a new boundary that is formed by line segments connecting the plurality of anchor indicators that represent the plurality of anchor points.

8. The method of claim 1, further comprising:
receiving user input that moves at least one anchor indicator of the plurality of anchor indicators in the graphical user interface;
adjusting the initial boundary to form a new boundary based on the user input; and
generating an adjusted image in which the new boundary is overlaid over the image; and
forming an input for an algorithm using the adjusted image.

9. The method of claim 1, wherein the image comprises a magnetic resonance imaging (MRI) image, a computed tomography (CT) image, an X-ray image, an ultrasound image, a positron-emission tomography (PET) image, a single-photon emission computerized tomography (SPECT) image, or an optical coherence tomography (OCT) image.

10. The method of claim 1, wherein the image is a two-dimensional image or a three-dimensional image.

11. A method comprising:
receiving a plurality of images associated with a portion of anatomy of a subject;
performing segmentation using the plurality of images and a segmentation model to generate a plurality of segmented images;
extracting, for each segmented image of the plurality of segmented images, a plurality of boundary points for each initial boundary of a set of initial boundary associated with each segmented image, wherein the plurality of boundary points is associated with a sequential order for a selected two-dimensional plane corresponding to the plurality of images;
evaluating, for each segmented image of the plurality of segmented images, the plurality of boundary points for each initial boundary of the set of initial boundary associated with each segmented image according to the sequential order to select a plurality of anchor points from the plurality of boundary points for each initial boundary of the set of initial boundary identified in each image,
wherein the evaluating comprises:
determining that a first boundary point in the plurality of boundary points is a first anchor point;
determining that a last boundary point in the plurality of boundary points is a last anchor point; and
determining that a current boundary point of the plurality of boundary points being evaluated is a next anchor point in the plurality of anchor points when at least one perpendicular distance of a set of perpendicular distances computed for a corresponding set of middle boundary points with respect to a line extending between a previous anchor point and the current boundary point is greater than a selected threshold; and
generating an anchor point image for display in a graphical user interface on a display device, wherein the anchor point image includes a plurality of anchor indicators that represent the plurality of anchor points and a plurality of line segments connecting the plurality of anchor indicators.

12. The method of claim 11, wherein the segmentation model comprises a machine learning model and further comprising:
receiving user input that adjust a position of at least one of the plurality of anchor indicators;
adjusting an original position of at least one anchor point in the plurality of anchor points corresponding to the at least one of the plurality of anchor indicators to form final positions for the plurality of anchor points; and
modifying a corresponding segmented image of the plurality of segmented images using the final positions of the plurality of anchor points to form an adjusted segmented image for use in retraining the segmentation model.

13. A system including one or more computing devices, comprising:
one or more non-transitory computer-readable storage media including instructions; and
one or more processors coupled to the one or more storage media, the one or more processors configured to execute the instructions to:
receive an image associated with a portion of anatomy of a subject;
extract a plurality of boundary points for an initial boundary associated with a corresponding region of pixels in the image, wherein the plurality of boundary points is associated with a sequential order for a selected two-dimensional plane corresponding to the image;
evaluate the plurality of boundary points according to the sequential order to select a plurality of anchor points from the plurality of boundary points, wherein the evaluating comprises:
determining that a first boundary point in the plurality of boundary points is a first anchor point; and
determining that a current boundary point of the plurality of boundary points being evaluated is a next anchor point in the plurality of anchor points when at least one perpendicular distance computed for a portion of the initial boundary located between a previous anchor point and the current boundary point with respect to a line extending between the previous anchor point and the current boundary point is greater than a selected threshold; and
generate an anchor point image for display in a graphical user interface on a display device, wherein the anchor point image includes a plurality of anchor indicators that represent the plurality of anchor points.

14. The system of claim 13, wherein the one or more processors is configured to execute the instructions to determine that the current boundary point being evaluated is not a next anchor point in the plurality of anchor points when no boundary points are present along the initial boundary between the previous anchor point and the current boundary point being evaluated.

15. The system of claim 13, wherein the one or more processors is further configured to execute the instructions to:
receive user input that moves at least one anchor indicator of the plurality of anchor indicators in the graphical user interface;
adjust an original position of at least one anchor point in the plurality of anchor points that corresponds to the at least one anchor indicator based on the user input to form final positions for the plurality of anchor points; and
generate adjustment data based on the user input, wherein the adjustment data includes an adjusted segmented image in which at least some pixels of a segmented image are reclassified to form the adjusted segmented image based on the final positions of the plurality of anchor points.

16. The system of claim 15, wherein the one or more processors is further configured to execute the instructions to:
retrain a segmentation model that comprises a machine learning model using the adjusted segmented image.

17. The system of claim 13, wherein the one or more processors is further configured to execute the instructions to:
determine that a last boundary point in the plurality of boundary points is a last anchor point.

18. The system of claim 13, wherein the one or more processors is further configured to execute the instructions to:
compute a perpendicular distance between each middle boundary point of the plurality of boundary points located between the previous anchor point and the current boundary point and a line extending between the previous anchor point and the current boundary point to form a set of perpendicular distances;
determine that at least one perpendicular distance of the set of perpendicular distances is greater than the selected threshold; and
determine that the current boundary point is to be the next anchor point in response to a determination that the at least one perpendicular distance of the set of perpendicular distances is greater than the selected threshold.

19. The system of any claim 13, wherein the selected threshold is selected to be between 1 and 10 pixel units.

20. The system of claim 13, wherein the anchor point image displayed in the graphical user interface further includes a new boundary that is formed by line segments connecting the plurality of anchor indicators that represent the plurality of anchor points.
